(19) United States
(12) Patent Application Publication
(10) Pub. No.: US 2026/0325511 A1
(43) Pub. Date: Oct. 22, 2026

(54) METHODS OF USE OF A LIPOSOMAL COMPOSITION COMPRISING AN ATR INHIBITOR

(71) Applicant: Jazz Pharmaceuticals Therapeutics, Inc., Palo Alto, CA (US)

(72) Inventors: Leon WAN, Vancouver (CA); Shyam GARG, Richmond (CA); Robert HAUPTSCHEIN, Ewing, NJ (US); Philippe LEGROS, Burnaby (CA); Paul TARDI, Surrey (CA)

(21) Appl. No.: 19/485,961

(22) PCT Filed: Jul. 12, 2024
(86) PCT No.: PCT/US2024/037864

§ 371 Date: Nov. 19, 2025

Related U.S. Application Data
(60) Provisional application No. 63/513,749, filed on Jul. 14, 2023.

(51) Int. Cl. A61K 31/4965; A61K 9/127; A61K 31/282; A61K 31/4745; A61K 31/497; A61K 31/7068; A61K 33/243; A61P 35/00

(52) U.S. Cl. CPC A61K 31/4965; A61K 9/127; A61K 31/282; A61K 31/4745; A61K 31/497; A61K 31/7068; A61K 33/243; A61P 35/00

(57) ABSTRACT

The present disclosure generally relates to liposomal compositions comprising an ATR inhibitor and uses thereof for treating various diseases and disorders. In one aspect, the use relates to methods of treating cancer, such as pancreatic cancer, breast cancer, and lung cancer.

### CROSS REFERENCE TO RELATED APPLICATIONS

This application claims the benefit of U.S. Provisional Application No. 63/513,749, filed Jul. 14, 2023, the disclosure of which is incorporated by reference herein in its entirety.

### FIELD OF THE INVENTION

The present disclosure generally relates to use of a liposomal composition comprising an ATR inhibitor and uses thereof for treating various diseases and disorders. In one aspect, the use relates to methods of treating cancer, such as pancreatic cancer, breast cancer, and lung cancer.

### BACKGROUND OF THE INVENTION

The ataxia-telangiectasia and Rad3-related (ATR) kinase is a serine/threonine protein kinase believed to be involved in the cellular DNA damage repair processes and cell cycle signaling. ATR kinase acts with ATM (“ataxia telangiectasia mutated”) kinase and other proteins to regulate a cell's response to DNA damage, commonly referred to as the DNA Damage Response (“DDR”). The DDR is believed to stimulate DNA repair, promote survival and stalls cell cycle progression by activating cell cycle checkpoints, which provide time for repair. Without the DDR, cells are much more sensitive to DNA damage and readily die from DNA lesions induced by endogenous cellular processes such as DNA replication or exogenous DNA damaging agents commonly used in cancer therapy.

The disruption of ATR function (e.g., by gene deletion) has been shown to promote cancer cell death both in the absence and presence of DNA damaging agents. Mutations of ATR have been linked to cancers of the stomach and endometrium, and lead to increased sensitivity to ionizing radiation and abolished cell cycle checkpoints. ATR is essential for the viability of somatic cells, and deletion of ATR has been shown to result in loss of damage checkpoint responses and cell death. See Cortez et al., Science 294:1713-1716 (2001). ATR is also essential for the stability of fragile sites, and low ATR expression in Seckel syndrome patients results in increased chromosomal breakage following replication stress. See Casper et al., Am. J. Hum. Genet 75:654-660 (2004). The replication protein A (RPA) complex recruits ATR, and its interacting protein ATRIP, to sites of DNA damage, and ATR itself mediates the activation of the CHK 1 signaling cascade. See Zou et al., Science 300:1542-1548 (2003). ATR, like its related checkpoint kinase ATM, phosphorylates RAD17 early in a cascade that is critical to for checkpoint signaling in DNA-damaged cells. See Bao et al., Nature 411:969-974 (2001). It is believed that ATR is particularly essential in the early mammalian embryo, to sense incomplete DNA replication and prevent mitotic catastrophe.

While novel ATR inhibitors (ATRi) are gaining traction in clinical trials, dose-limiting toxicities such as anemia has impeded or prevented further development. See Roulston et al., Mol. Cancer Ther. 21 (2022) 245-256; Fang et al., Cancer Cell 35 (2019) 851-867.e7. Thus, there is a need for effectively delivering an ATRi to treat cancer such that it eliminates dose-limiting toxicities.

U.S. Pat. Nos. 10,570,119, 11,028,076, and 11,787,781 discloses ATR inhibitors, each of the disclosures is hereby incorporated by reference in its entirety.

### SUMMARY OF THE INVENTION

The present disclosure provides liposomal compositions comprising (i) a compound of formula (I) or a pharmaceutically acceptable salt, tautomer, or stereoisomer thereof, and (ii) a liposome comprising a phospholipid and a cholesterol, which is useful in treating cancer.

In embodiments, the present disclosure provides a method of treating cancer in a subject in need thereof, comprising administering a liposomal composition comprising:
(i) a compound of formula (I):

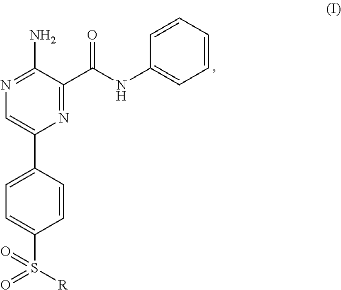

or a pharmaceutically acceptable salt thereof, wherein
R is a moiety comprising an amino group with a pKₐ of greater than 7.0; and
(ii) a liposome comprising a phospholipid and a cholesterol.

In embodiments, provided herein are methods of treating cancer in a mammal in need thereof, comprising administering a liposomal composition comprising:
(i) a compound of formula (I):

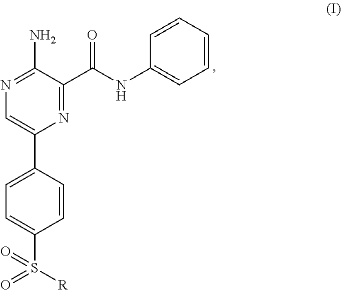

or a pharmaceutically acceptable salt thereof, wherein
R is a moiety comprising an amino group with a pKₐ of greater than 7.0; and
(ii) a liposome comprising a phospholipid and a cholesterol, with the proviso that the cancer is not lung cancer or breast cancer.

In embodiments, the present disclosure provides a method of treating cancer in a mammal in need thereof, comprising administering a liposomal composition comprising:
(i) a compound of formula (I):

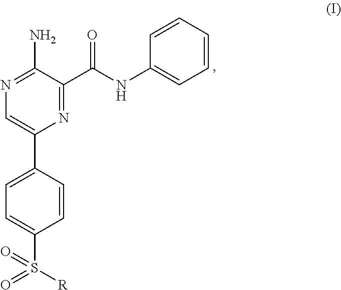

or a pharmaceutically acceptable salt thereof, wherein
R is a moiety comprising an amino group with a pKₐ of greater than 7.0; and
(ii) a liposome comprising a phospholipid and a cholesterol;
with the proviso that the phospholipid is not hydrogenated soy phosphatidylcholine (HSPC).

The present disclosure also provides liposomal compositions comprising (i) a compound of formula (I) or a pharmaceutically acceptable salt, tautomer, or stereoisomer thereof, and (ii) a liposome comprising a phospholipid, a cholesterol, and a PEG-lipid conjugate; wherein the composition is stable for at least 2 weeks.

The present disclosure also provides liposomal compositions comprising (i) a compound of formula (I) or a pharmaceutically acceptable salt, tautomer, or stereoisomer thereof, and (ii) a liposome comprising a phospholipid, a cholesterol, and a PEG-DSG, wherein the liposome comprises about 0.1 mol % to about 2 mol % PEG-DSG.

Each of the above composition is a “composition of the invention”.

The present disclosure also provides methods for treating cancer, comprising administering to a subject in need thereof an effective amount of the composition of the invention (each method being a “method of the invention”).

In embodiments, the subject is a mammal.

### BRIEF DESCRIPTION OF THE DRAWINGS

FIG. 1A shows levels of Compound A in plasma and in tumor after a single IV administration of liposomal and non-liposomal composition of Compound A in CRC Colo205 tumor model mice.

FIG. 1B shows levels of Compound A in plasma and in tumor after a single IV administration at 40 mg/kg IV of liposomal (lipo-G Comp. A, Lipo-E Comp. A) and non-liposomal composition of Compound A in CRC Colo205 tumor model mice. Sampling was conducted 24 hours after last dose and study was run in duplicate (2 mice per treatment group).

FIG. 2A shows the change in median tumor volume for different study groups as described in Example 3. FIG. 2B is a magnified view of a portion of FIG. 2A.

FIG. 3 shows % change in individual tumor volume at day 48 compared to day 0 in the study described in Example 3.

FIG. 4A shows the change in mean tumor volume for different study groups as described in Example 4A.

FIG. 4B shows the change in mean tumor volume for different study groups as described in Example 4B.

FIG. 5A shows the change in mean tumor volume for different study groups as described in Example 5.

FIG. 5B shows % tumor volume change for mice in the different study groups post last dose as described in Example 5.

FIG. 6 shows the change in mean tumor volume for different study groups as described in Example 6, Table 8.

FIG. 7 shows the % tumor volume change for mice in the different study groups as described in Example 6.

FIG. 8 shows reticulocyte count for different study groups disclosed in Example 6. Samples were collected 72 hours after the 4th dose for liposomal G compound A groups. For ceralasertib and berzosertib groups samples were collected 2 hours after PO AM dose on the day of collection. *p-value<0.05; **p-value<0.01, I=irinotecan 20 mg/kg IP.

FIG. 9 shows red blood cell (RBC) count for different study groups disclosed in Example 6. Samples were collected 72 hours after the 4th dose for liposomal G compound A groups. For ceralasertib and berzosertib groups samples were collected 2 hours after PO AM dose on the day of collection. *p-value<0.05; **p-value<0.01, I=irinotecan 20 mg/kg IP.

FIG. 10 shows HB count for different study groups disclosed in Example 6. Samples were collected 72 hours after the 4th dose for liposomal G compound A groups. For ceralasertib and berzosertib groups samples were collected 2 hours after PO AM dose on the day of collection. *p-value<0.05; **p-value<0.01, I=irinotecan 20 mg/kg IP.

FIG. 11 shows lymphocyte count for different study groups disclosed in Example 6.

FIG. 12 shows mean tumor volume for different study groups as described in Example 6, Table 9.

FIG. 13A shows the probability of survival for different study groups as described in Example 7, Table 10.

FIG. 13B the % tumor volume change for mice in the different study groups as described in Example 7, Table 10.

FIG. 14 shows shows the change in OVCAR8 tumor volume (mm³) after treatment with a combination of liposomal Compound A (in Formulation G) and irinotecan administered at 50 mg/kg in comparison to empty liposomes (Formulation G, or Lipo. G; “Vehicle”), liposomal Compound A (in Formulation G) administered at 80 mg/kg, and free irinotecan administered at 50 mg/kg using a OVCAR8 tumor model.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “an ATR inhibitor” refers to one or more ATR inhibitor or at least one ATR inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The terms “pharmaceutical combination,” “therapeutic combination” or “combination” as used herein, refers to a single composition or dosage form comprising at least two therapeutically active agents (such as, ATRi and irinotecan), or separate composition or dosage forms comprising at least two therapeutically active agents together or separately for use in a combination therapy. For example, one therapeutically active agent may be formulated into one composition or dosage form and the other therapeutically active agent may be formulated into a single or different compositions or dosage forms; these can be given together or separately. For example, one therapeutically active agent (e.g., ATRi) may be formulated into a first liposomal formulation whereas the second therapeutically active agent (such as irinotecan) may be in a free form or formulated into a second liposomal formulation. In some embodiments, the combination includes one or more kits.

The term “combination therapy” as used herein refers to a first therapy that comprises an ATRi in conjunction with a second therapy that is useful for treating, ameliorating, and/or delaying the disease or condition. For example, a second therapy can be with an antineoplastic agent, such as irinotecan.

Administration in “conjunction with” another therapeutically active agent includes administration in the same or different composition(s) and/or combinations, either sequentially, simultaneously, or continuously, through the same or different routes. In some embodiments, two or more therapeutically active agents that are used in combination therapy are on the same or different dosing schedule which can start at the same time or separately. For example, in one embodiment of the method of the invention, ATRi can be administered once a week and another therapeutically active agent can be administered once every three days.

The term “liposome” as used herein means vesicles comprised of one or more concentrically ordered lipid bilayers encapsulating an aqueous phase. Formation of such vesicles requires the presence of vesicle-forming lipids (also referred to as “liposome lipids” herein) which are amphipathic lipids capable of either forming or being incorporated into a bilayer structure. The latter term includes lipids that are capable of forming a bilayer by themselves or when in combination with another lipid or lipids. An amphipathic lipid is incorporated into a lipid bilayer by having its hydrophobic moiety in contact with the interior, hydrophobic region of the membrane bilayer and its polar head moiety oriented toward an outer, polar surface of the membrane. Hydrophilicity arises from the presence of functional groups such as hydroxyl, phosphato, carboxyl, sulfato, amino or sulfhydryl groups. Hydrophobicity results from the presence of a long chain of aliphatic hydrocarbon groups.

The term “non-liposomal,” “liposome-free,” “free drug (fd)” or “free” formulation or active ingredient as used herein refers to a therapeutically active ingredient/agent (such as an ATRi) that is not encapsulated in a liposome (or other nanoparticulate delivery vehicle) and is administered, in a formulation or solution that is not intended to control the release rate of the therapeutically active ingredient/agent.

As used herein, a “subject” can be a human, non-human primate, mammal, rat, mouse, cow, horse, pig, sheep, goat, dog, cat and the like. The subject can be suspected of having or at risk for having a cancer, including but not limited to colorectal cancer and melanoma.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

“Alkyl” or “alkyl group” refers to a fully saturated, straight or branched hydrocarbon chain group, and which is attached to the rest of the molecule by a single bond. Alkyls comprising any number of carbon atoms, including but not limited to from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈alkyl. A C₁-C₈alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₈alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-Nonyl, n-decyl, n-undecyl, and n-dodecyl. Unless stated otherwise specifically in the specification, an alkyl group can be optionally substituted.

“Alkylene” refers to a fully saturated, straight or branched hydrocarbon chain group, and which is attached to the rest of the molecule by two single bonds (divalent)—i.e., an alkyl group as defined above that has one additional point of attachment. Unless stated otherwise specifically in the specification, an alkylene group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing groups can also be optionally substituted with one or more of the above groups.

### Compounds Useful in the Compositions and the Methods of the Invention

The present disclosure relates to liposomal compositions comprising a compound of formula (I) or a pharmaceutically acceptable salt, tautomer, or stereoisomer thereof.

The composition of the invention comprises a compound of formula (I):

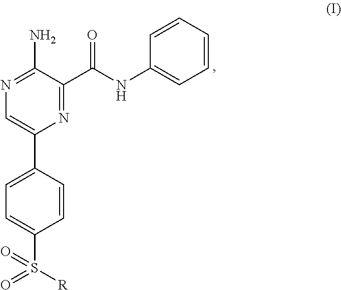

or a pharmaceutically acceptable salt thereof, wherein R is a moiety comprising an amino group with a pKₐ of greater than 7.0. In some embodiments

In some embodiments, the compound of formula (I) or a pharmaceutically acceptable salt thereof is an ATR inhibitor.

In some embodiments of the compound of formula (I), R is -alkylene-NR^{a}R^{b}, -(alkylene)-(heterocyclyl)-(alkyl), -(alkylene)-(heterocyclyl)-(alkylene)-NR^{a}R^{b}, -(alkylene)-(heterocyclyl)-NR^{a}R^{b}, or —NR^{a}R^{b}-(alkyl), wherein the heterocyclyl contains at least one nitrogen as a ring atom; and R^{a} and R^{b} are each independently H or alkyl. In some embodiments of the compound of formula (I), R is —C₁-C₄ alkylene-NR^{a}R^{b}, —(C₁-C₆ alkylene)-(heterocyclyl)-(C₁-C₆ alkyl), —(C₁-C₆ alkylene)-(heterocyclyl)-(C₁-C₆ alkylene)-NR^{a}R^{b}, —(C₁-C₆ alkylene)-(heterocyclyl)-NR^{a}R^{b}, or —NR^{a}R^{b}—(C₁-C₆ alkyl), wherein the heterocyclyl contains at least one nitrogen as a ring atom; and R^{a} and R^{b} are each independently H or —C₁-C₆ alkyl.

In some embodiments of the compound of formula (I), R is —C₁-C₄ alkylene-NR^{a}R^{b}, —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, —(C₁-C₄ alkylene)-(heterocyclyl)-NR^{a}R^{b}, or —NR^{a}R^{b}—(C₁-C₄ alkyl), wherein the heterocyclyl contains at least one nitrogen as a ring atom; and R^{a} and R^{b} are each independently H or —C₁-C₄ alkyl. In some embodiments, the heterocyclyl is a divalent heterocycle containing one nitrogen as a ring atom and optionally one additional heteroatom selected from N, S, or O. In some embodiments, the heterocyclyl is a divalent heterocycle containing one nitrogen as a ring atom. In some embodiments, the heterocyclyl is a divalent heterocycle containing two nitrogen atoms as ring atoms. In some embodiments, the heterocyclyl is a divalent 5- to 7-membered heterocycle containing at least one nitrogen as a ring atom. In some embodiments, the heterocyclyl is a divalent 6-membered heterocycle containing at least one nitrogen as a ring atom. In some embodiments, the heterocyclyl is a divalent 6-membered heterocycle containing at least one nitrogen as a ring atom and optionally one additional heteroatom selected from N, S, or O. In some embodiments, the heterocyclyl is a divalent 6-membered heterocycle containing one nitrogen as a ring atom. In some embodiments, the heterocyclyl is a divalent 6-membered heterocycle containing two nitrogen atoms as ring atoms. In some embodiments, the heterocyclyl is a divalent 5- to 7-membered saturated heterocycle containing at least one nitrogen as a ring atom. In some embodiments, the heterocyclyl is a divalent 6-membered saturated heterocycle containing at least one nitrogen as a ring atom. In some embodiments, the heterocyclyl is a divalent 6-membered saturated heterocycle containing at least one nitrogen as a ring atom and optionally one additional heteroatom selected from N, S, or O. In some embodiments, the heterocyclyl is a divalent 6-membered saturated heterocycle containing one nitrogen as a ring atom. In some embodiments, the heterocyclyl is a divalent 6-membered saturated heterocycle containing two nitrogen atoms as ring atoms.

In some embodiments of the compound of formula (I), R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

In some embodiments of the compound of formula (I), R is:
a)

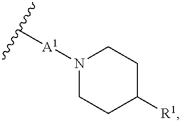

wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkyl)-NR^{a}R^{b}, wherein R^{a} and R^{b} are each independently H or —C₁-C₄ alkyl;
c) —(C₁-C₈ alkyl)-NR^{a}R^{b}, wherein R^{a} and R^{b} are each independently H or C₁-C₄ alkyl; or
d)

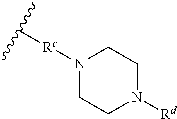

wherein R^{c} is —C₁-C₄ alkylene and R^{d} is —C₁-C₄ alkyl.

In some embodiments of the compound of formula (I), R is

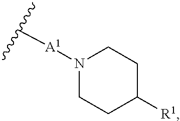

wherein A¹ is absent or alkylene, and R¹ is -(alkylene)-NR^{a}R^{b}, wherein R^{a} and R^{b} are each independently H or alkyl. In some embodiments, R is

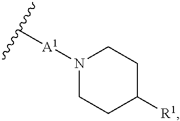

wherein A¹ is absent or C₁-C₆ alkylene, and R¹ is —(C₁-C₆ alkylene)-NR^{a}R^{b}, wherein R^{a} and R^{b} are each independently H or —C₁-C₆ alkyl. In some embodiments, R is

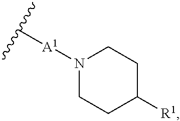

wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl. In some embodiments, A¹ is C₁-C₄ alkylene. In some embodiments, A¹ is a straight or branched C₁-C₄ alkylene. In some embodiments, A¹ is —CH₂—, —CH₂CH₂—, or —CH₂CH₂CH₂—. In some embodiments, A¹ is —CH₂CH₂—.

In some embodiments of the compound of formula (I), R is —N(R^{a})(alkylene)-NR^{a}R^{b}, wherein R^{a} and R^{b} are each independently H or alkyl. In some embodiments, R is —N(R^{a})(C₁-C₆ alkylene)-NR^{a}R^{b}, wherein R^{a} and R^{b} are each independently H or —C₁-C₆ alkyl. In some embodiments, R is —N(R^{a})(C₁-C₄ alkylene)-NR^{a}R^{b}, wherein R^{a} and R^{b} are each independently H or —C₁-C₄ alkyl. In some embodiments, R is —N(R^{a})(C₁-C₄ alkylene)-NR^{a}R^{b}, wherein C₁-C₄ alkylene is straight or branched; and R^{a} and R^{b} are each independently H or —C₁-C₄ alkyl. In some embodiments, R is —N(H)(C₁-C₄ alkylene)-NR^{a}R^{b}, wherein R^{a} and R^{b} are each independently H or —C₁-C₄ alkyl. In some embodiments, R is —NHCH₂—NR^{a}R^{b}, —NHCH₂CH₂—NR^{a}R^{b}, or —NHCH₂CH₂CH₂—NR^{a}R^{b}, or wherein R^{a} and R^{b} are each independently H or —C₁-C₄ alkyl. In some embodiments, R is —NHCH₂—NR^{a}R^{b}, —NHCH₂CH₂—NR^{a}R^{b}, or —NHCH₂CH₂CH₂—NR^{a}R^{b}, or wherein R^{a} and R^{b} are each independently-C₁-C₄ alkyl. In some embodiments, R is —NHCH₂—NR^{a}R^{b}, —NHCH₂CH₂—NR^{a}R^{b}, or —NHCH₂CH₂CH₂—NR^{a}R^{b}, or wherein R^{a} and R^{b} are each independently methyl, ethyl, or propyl. In some embodiments, R is —NHCH₂CH₂—NR^{a}R^{b}, wherein R^{a} and R^{b} are each methyl.

In some embodiments of the compound of formula (I), R is-(alkylene)-NR^{a}R^{b}, wherein R^{a} and R^{b} are each independently H or alkyl. In some embodiments, R is —(C₁-C₁₀ alkylene)-NR^{a}R^{b}, wherein R^{a} and R^{b} are each independently H or C₁-C₆ alkyl. In some embodiments, R is —(C₁-C₈ alkylene)-NR^{a}R^{b}, wherein R^{a} and R^{b} are each independently H or C₁-C₄ alkyl. In some embodiments, R is —(C₁-C₈ alkylene)-NR^{a}R^{b} wherein C₁-C₈ alkylene is straight or branched; and R^{a} and R^{b} are each independently H or C₁-C₄ alkyl. In some embodiments, R is (C₁-C₄ alkylene)-NR^{a}R^{b}, wherein R^{a} and R^{b} are each independently H or C₁-C₄ alkyl. In some embodiments, R is —(C₁-C₄ alkylene)-NR^{a}R^{b} wherein C₁-C₄ alkylene is straight or branched; and R^{a} and R^{b} are each independently H or C₁-C₄ alkyl. In some embodiments, R is —(C₁-C₄ alkylene)-NR^{a}R^{b}, wherein R^{a} and R^{b} are each independently H or C₁-C₄ alkyl. In some embodiments, R is —CH₂ NR^{a}R^{b}, —CH₂CH₂NR^{a}R^{b}, —CH₂CH₂CH₂NR^{a}R^{b}, or —CH(CH₃)CH₂NR^{a}R^{b}, wherein R^{a} and R^{b} are each independently H or C₁-C₄ alkyl. In some embodiments, R is —CH₂NR^{a}R^{b}, —CH₂CH₂NR^{a}R^{b}, —CH₂CH₂CH₂NR^{a}R^{b}, or —CH(CH₃)CH₂NR^{a}R^{b}, wherein R^{a} and R^{b} are each independently C₁-C₄ alkyl. In some embodiments, R is —CH₂NR^{a}R^{b}, —CH₂CH₂NR^{a}R^{b}, —CH₂CH₂CH₂NR^{a}R^{b}, or —CH(CH₃)CH₂NR^{a}R^{b}, wherein R^{a} and R^{b} are each independently methyl, ethyl, or propyl. In some embodiments, R is —CH₂NR^{a}R^{b}, —CH₂CH₂NR^{a}R^{b}, —CH₂CH₂CH₂NR^{a}R^{b}, or —CH(CH₃)CH₂NR^{a}R^{b}, wherein R^{a} and R^{b} are each independently methyl or ethyl. In some embodiments, R is —CH(CH₃)CH₂NR^{a}R^{b}, wherein R^{a} and R^{b} are each independently methyl, ethyl, or propyl. In some embodiments, R is —CH₂NR^{a}R^{b}, —CH₂CH₂NR^{a}R^{b}, —CH₂CH₂CH₂NR^{a}R^{b}, or —CH(CH₃)CH₂NR^{a}R^{b}, wherein R^{a} and R^{b} are each methyl. In some embodiments, R is —CH₂NR^{a}R^{b}, —CH₂CH₂NR^{a}R^{b}, —CH₂CH₂CH₂NR^{a}R^{b}, or —CH(CH₃)CH₂NR^{a}R^{b}, wherein R^{a} and R^{b} are each independently methyl, ethyl, or propyl. In some embodiments, R is —CH₂CH₂NR^{a}R^{b}, wherein R^{a} and R^{b} are each ethyl.

In some embodiments of the compound of formula (I), R is

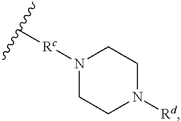

wherein R^{e} is alkylene and R^{d} is alkyl. In some embodiments, R is

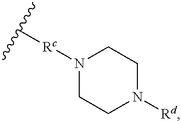

wherein R^{c} is —C₁-C₆ alkylene and R^{d} is —C₁-C₆ alkyl. In some embodiments, R is

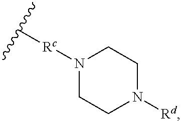

wherein R^{c} is —C₁-C₄ alkylene and R^{d} is —C₁-C₄ alkyl. In some embodiments, R is

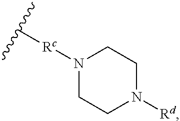

wherein R^{c} is —C₁-C₄ alkylene wherein-C₁-C₄ alkylene is straight or branched, and R^{d} is —C₁-C₄ alkyl. In some embodiments, R is

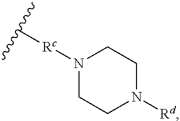

wherein R^{c} is —CH₂—, —CH₂CH₂—, or —CH₂CH₂CH₂— and R^{d} is methyl, ethyl, or propyl. In some embodiments, R is

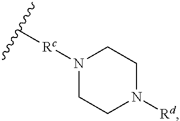

wherein R^{c} is —CH₂CH₂— and R^{d} is methyl.

In some embodiments, the compound of formula (I) is selected from Table A, or a pharmaceutically acceptable salt thereof. In some embodiments, the compound of formula (I) is Compound 1, Compound 2, Compound 3, Compound 4, Compound 5, or Compound 6, or a pharmaceutically acceptable salt thereof.

TABLE A
Compounds of Formula (I)

| | |
|---|---|
| 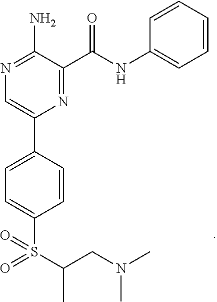 [SMILES: CC(CN(C)C)S(=O)(=O)c1ccc(-c2cnc(N)c(C(=O)Nc3ccccc3)n2)cc1] | Compound 1 |
| 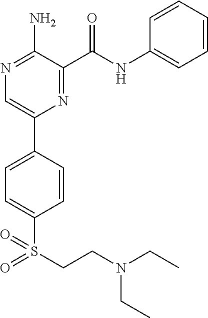 [SMILES: CCN(CC)CCS(=O)(=O)c1ccc(-c2cnc(N)c(C(=O)Nc3ccccc3)n2)cc1] | Compound 2 |
| 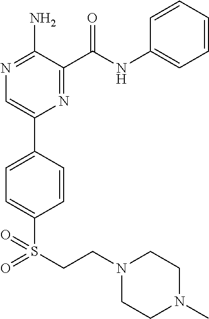 [SMILES: CN1CCN(CCS(=O)(=O)c2ccc(-c3cnc(N)c(C(=O)Nc4ccccc4)n3)cc2)CC1] | Compound 3 |
| 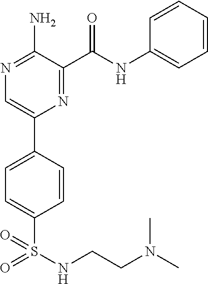 [SMILES: CN(C)CCNS(=O)(=O)c1ccc(-c2cnc(N)c(C(=O)Nc3ccccc3)n2)cc1] | Compound 4 |
| 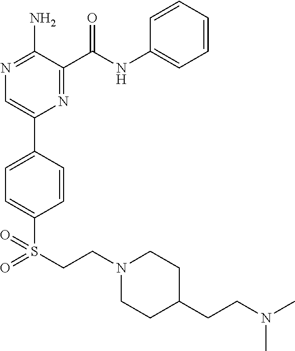 [SMILES: CN(C)CCC1CCN(CCS(=O)(=O)c2ccc(-c3cnc(N)c(C(=O)Nc4ccccc4)n3)cc2)CC1] | Compound 5 |
| 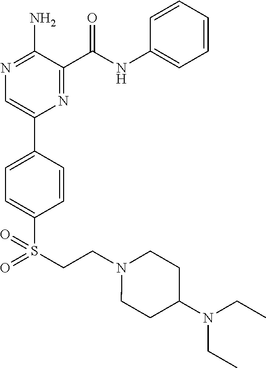 [SMILES: CCN(CC)C1CCN(CCS(=O)(=O)c2ccc(-c3cnc(N)c(C(=O)Nc4ccccc4)n3)cc2)CC1] | Compound 6 |

### Compositions of the Invention

The present disclosure also relates to liposomal composition comprising a compound of formula (I) or a pharmaceutically acceptable salt thereof. In some embodiments, the liposomal composition comprises a compound of formula (I) or a pharmaceutically acceptable salt thereof and a liposome comprising a phospholipid and a cholesterol. In some embodiments, the liposomal composition comprises a compound of formula (I) or a pharmaceutically acceptable salt thereof and a liposome comprising a phospholipid, a cholesterol, and a PEG-lipid conjugate. In some embodiments, the liposomal composition further comprises a pharmaceutically acceptable carrier or excipient. In some embodiments, the liposomal composition is stable for at least 2 weeks. In some embodiments, the liposome of the liposomal composition comprises about 0.1 mol % to about 2 mol % PEG-DSG.

In embodiments, the liposomal composition comprising a compound of formula (I) or a pharmaceutically acceptable salt thereof, comprises the compound of formula (I) or a pharmaceutically acceptable salt thereof as the only pharmaceutically active agent in the liposomal composition.

In embodiments, the present disclosure provides a liposomal composition comprising:
(i) a compound of formula (I):

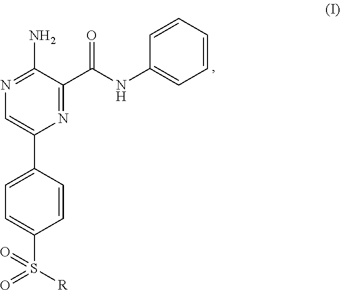

or a pharmaceutically acceptable salt thereof, wherein
R is a moiety comprising an amino group with a pKₐ of greater than 7.0; and
(ii) a liposome comprising a phospholipid and a cholesterol.

In embodiments, the liposomal compositions disclosed herein are useful for treating cancer.

In embodiments, the present disclosure provides a liposomal composition comprising:
(i) a compound of formula (I):

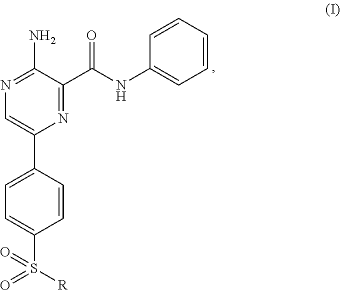

or a pharmaceutically acceptable salt thereof, wherein
R is a moiety comprising an amino group with a pKₐ of greater than 7.0; and
(ii) a liposome comprising 1,2-distearoyl-sn-glycero-3-phosphocholine (DSPC) and a cholesterol.

In embodiments, the present disclosure provides a liposomal composition comprising:
(i) a compound of formula (I):

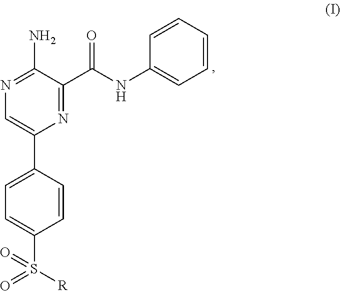

or a pharmaceutically acceptable salt thereof, wherein
R is a moiety comprising an amino group with a pKₐ of greater than 7.0; and
(ii) a liposome comprising a phospholipid, a cholesterol, and a PEG-lipid conjugate; wherein the composition is stable for at least two weeks.

In some embodiments, the present disclosure provides a liposomal composition comprising:
(i) a compound of formula (I):

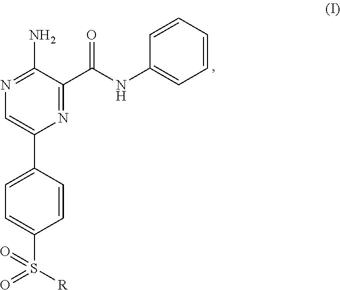

or a pharmaceutically acceptable salt thereof, wherein
R is a moiety comprising an amino group with a pKₐ of greater than 7.0; and
(ii) a liposome comprising a phospholipid, a cholesterol, and a PEG-lipid conjugate; wherein the liposome comprises about 0.1 mol % to about 2.5 mol % of the PEG-lipid conjugate.

In some embodiments, the present disclosure provides a liposomal composition comprising:
(i) a compound of formula (I):

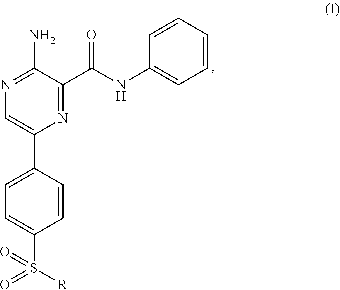

or a pharmaceutically acceptable salt thereof, wherein
R is a moiety comprising an amino group with a pKₐ of greater than 7.0; and
(ii) a liposome comprising a phospholipid, a cholesterol, and a PEG-lipid conjugate; wherein the liposome comprises about 0.1 mol % to about 2 mol % of the PEG-lipid conjugate.

In some embodiments, the PEG-lipid conjugate is PEG-DSG.

In some embodiments, the present disclosure provides a liposomal composition comprising:
(i) a compound of formula (I):

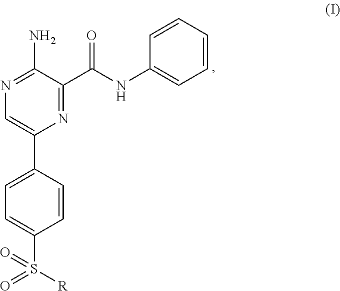

or a pharmaceutically acceptable salt thereof, wherein
R is a moiety comprising an amino group with a pKₐ of greater than 7.0; and
(ii) a liposome comprising a phospholipid, a cholesterol, and a PEG-DSG;
   wherein the liposome comprises about 0.1 mol % to about 2 mol % of the PEG-DSG.

In some embodiments, the composition does not have visible precipitates for at least 2 weeks after preparation of the composition. In some embodiments, the composition does not have visible precipitates for at least 1 month, at least 2 months, at least 3 months, at least 4 months, or at least 5 months after preparation of the composition. In some embodiments, the composition does not have visible precipitates for at least 6 months after preparation of the composition. In some embodiments, the composition does not have visible precipitates for at least 12 months after preparation of the composition.

In some embodiments, the composition of the invention further comprises an additional therapeutic agent. The additional pharmaceutically active agent may be an anti-cancer agent.

In some embodiments, the present disclosure provides a pharmaceutical combination comprising a composition comprising a compound of formula (I) or a pharmaceutically acceptable salt thereof, and an additional pharmaceutically active agent. In embodiments, the composition comprising a compound of formula (I) or a pharmaceutically acceptable salt thereof, is a liposomal composition (e.g., any disclosed herein).

In some embodiments, the pharmaceutical combinations of the invention provide therapeutic synergy between the compound of formula (I) or a pharmaceutically acceptable salt thereof, and an additional pharmaceutically active agent.

In embodiments, the additional therapeutic agent that may be used in combination include DNA damaging agents such as carboplatin, cisplatin, cyclophosphamide, doxorubicin, daunorubicin, epirubicin, mitomycin C, mitoxantrone; DNA repair inhibitors including 5-fluorouracil (5-FU) or FUDR, gemcitabine and methotrexate; topoisomerase I inhibitors such as camptothecin, irinotecan and topotecan; S/G2 or G2/M checkpoint inhibitors such as bleomycin, docetaxel, doxorubicin, etoposide, paclitaxel, vinblastine, vincristine, vindesine and vinorelbine; G1/early-S checkpoint inhibitors; G2/M checkpoint inhibitors; receptor tyrosine kinase inhibitors such as genistein, trastuzumab, ZD1839; cytotoxic agents; apoptosis-inducing agents and cell cycle control inhibitors.

In embodiments, the additional pharmaceutically active agent is an anti-cancer agent.

In embodiments, the additional pharmaceutically active agent is a camptothecin derivative such as topotecan or irinotecan or a pharmaceutically acceptable salt thereof.

In embodiments, the additional pharmaceutically active agent is a camptothecin derivative such as topotecan or irinotecan.

In some embodiments, the additional pharmaceutically active agent is irinotecan or a pharmaceutically acceptable salt thereof.

In some embodiments, the additional pharmaceutically active agent is irinotecan.

In embodiments, the additional pharmaceutically active agent is an alkylating agent, for example a platinum agent such as platinum coordination complexes e.g., cisplatin, carboplatin or oxaliplatin. In embodiments, the platinum agent is cisplatin or carboplatin.

In embodiments, the additional pharmaceutically active agent is gemcitabine.

In embodiments, the additional pharmaceutically active agent is cisplatin.

In embodiments, the present disclosure provides a pharmaceutical combination comprising a liposomal composition comprising Compound 1:

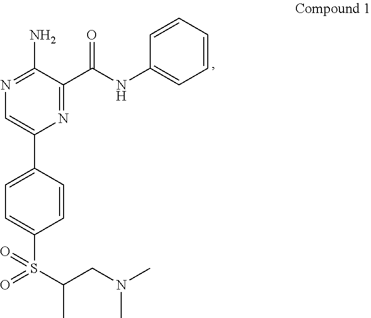 [SMILES: CC(CN(C)C)S(=O)(=O)c1ccc(-c2cnc(N)c(C(=O)Nc3ccccc3)n2)cc1]

or a pharmaceutically acceptable salt thereof and a chemotherapeutic agent, wherein the chemotherapeutic agent is in free form and wherein the liposomal composition comprises DSPC, cholesterol and a PEG-lipid conjugate.

In embodiments, the present disclosure provides a pharmaceutical combination comprising a liposomal composition comprising Compound 2:

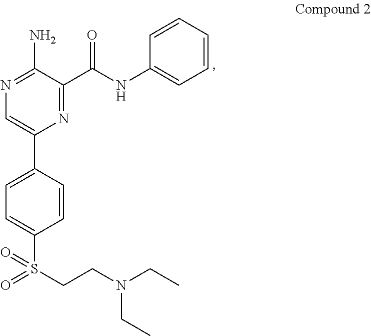 [SMILES: CCN(CC)CCS(=O)(=O)c1ccc(-c2cnc(N)c(C(=O)Nc3ccccc3)n2)cc1]

or a pharmaceutically acceptable salt thereof and a chemotherapeutic agent, wherein the chemotherapeutic agent is in free form and wherein the liposomal composition comprises DSPC, cholesterol and a PEG-lipid conjugate.

In embodiments, the present disclosure provides a pharmaceutical combination comprising a liposomal composition comprising Compound 3:

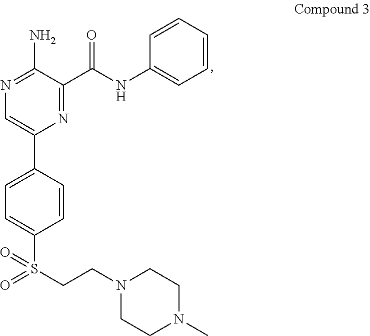 [SMILES: CN1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent, wherein the chemotherapeutic agent is in free form and wherein the liposomal composition comprises DSPC, cholesterol and a PEG-lipid conjugate.

In embodiments, the present disclosure provides a pharmaceutical combination comprising a liposomal composition comprising Compound 4:

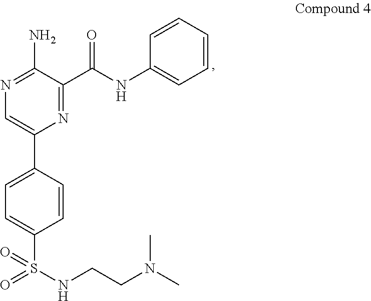 [SMILES: CN(C)CCNS(=O)(=O)c1ccc(-c2cnc(N)c(C(=O)Nc3ccccc3)n2)cc1]

or a pharmaceutically acceptable salt thereof and a chemotherapeutic agent, wherein the chemotherapeutic agent is in free form and wherein the liposomal composition comprises DSPC, cholesterol and a PEG-lipid conjugate.

In embodiments, the present disclosure provides a pharmaceutical combination comprising a liposomal composition comprising Compound 5:

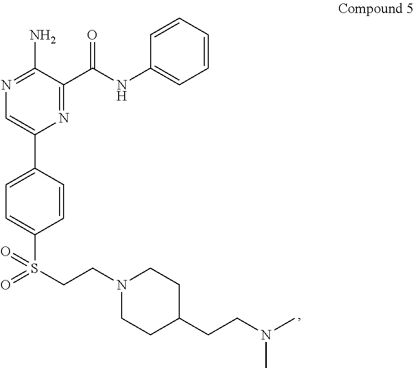 [SMILES: CN(C)CCC1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent, wherein the chemotherapeutic agent is in free form and wherein the liposomal composition comprises DSPC, cholesterol and a PEG-lipid conjugate.

In embodiments, the present disclosure provides a pharmaceutical combination comprising a liposomal composition comprising Compound 6:

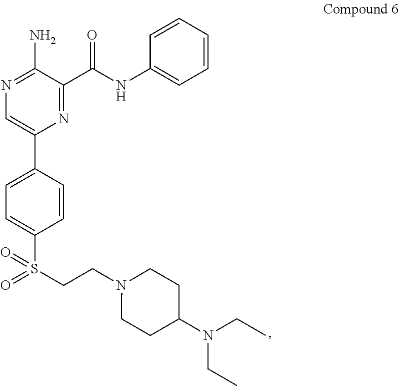 [SMILES: CCN(CC)C1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent, wherein the chemotherapeutic agent is in free form and wherein the liposomal composition comprises DSPC, cholesterol and a PEG-lipid conjugate.

In embodiments, the chemotherapeutic agent is irinotecan, topotecan, gemcitabine, cisplatin, or carboplatin.

In some embodiments, the additional therapeutically active agent is formulated in the same composition as the composition of the invention. In some embodiments, the additional therapeutically active agent is formulated separate from the composition of the invention. In some embodiments, the additional therapeutically active agent is formulated into a different liposomal composition. In embodiments, the additional therapeutically active agent is the only pharmaceutically active agent in the liposomal composition. In embodiments, the additional therapeutically active agent is formulated in a non-liposomal composition. In an embodiment, the additional therapeutically active agent is the only pharmaceutically active agent in the non-liposomal composition.

In some embodiments, the pharmaceutical composition (e.g., any pharmaceutical composition disclosed herein) is in a form of a cream, gel, lotion, solution or liquid, suspension, solid, syrup, powder or granule.

In some embodiments, the pharmaceutical composition (e.g., any pharmaceutical composition disclosed herein) is formulated for oral administration, topical administration, parenteral administration (including intravenous, intraarterial, intraperitoneal, subcutaneous, intramuscular, intravascular or infusion); rectal administration, or by inhalation (including aerosol). In some embodiments, the pharmaceutical composition (e.g., any pharmaceutical composition disclosed herein) is formulated for administration intravenously or intraperitoneally by a bolus injection or infusion.

Liposomal compositions can provide desirable pharmacokinetic properties for the compounds of formula (I) or a pharmaceutically acceptable salt thereof. The liposomes typically comprise vesicles containing one or more lipid bilayers enclosing an aqueous interior. Liposome compositions usually include liposomes in a medium, such as an aqueous fluid exterior to the liposome. Liposome lipids can include amphiphilic lipid components that, upon contact with aqueous medium, spontaneously form bilayer membranes, such as phospholipids, for example, phosphatidylcholines. Liposomes also can include membrane-rigidifying components, such as sterols, for example, cholesterol. In some embodiments, liposomes also include lipids conjugated to hydrophilic polymers, such as, PEG-lipid conjugates that may reduce the tendency of liposomes to aggregate and also have other beneficial effects.

In some embodiments, the liposomal composition comprises a compound of formula (I) or a pharmaceutically acceptable salt thereof encapsulated in a liposome. In some embodiments, the liposomal composition comprises a compound of formula (I) or a pharmaceutically acceptable salt thereof as the only pharmaceutically active agent. In some embodiments, the liposomal composition comprises a compound of formula (I) or a pharmaceutically acceptable salt thereof and an additional therapeutically active agent in the same composition.

In embodiments, the present disclosure provides a pharmaceutical combination comprising a liposomal composition comprising Compound 1:

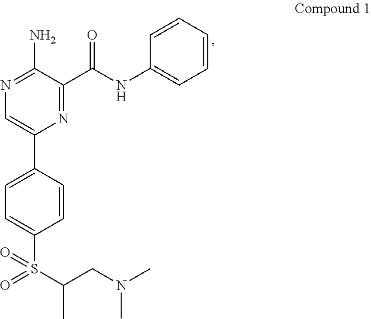 [SMILES: CC(CN(C)C)S(=O)(=O)c1ccc(-c2cnc(N)c(C(=O)Nc3ccccc3)n2)cc1]

or a pharmaceutically acceptable salt thereof and a chemotherapeutic agent.

In embodiments, the present disclosure provides a pharmaceutical combination comprising a liposomal composition comprising Compound 2:

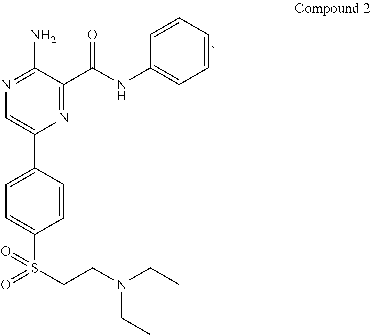 [SMILES: CCN(CC)CCS(=O)(=O)c1ccc(-c2cnc(N)c(C(=O)Nc3ccccc3)n2)cc1]

or a pharmaceutically acceptable salt thereof and a chemotherapeutic agent.

In embodiments, the present disclosure provides a pharmaceutical combination comprising a liposomal composition comprising Compound 3:

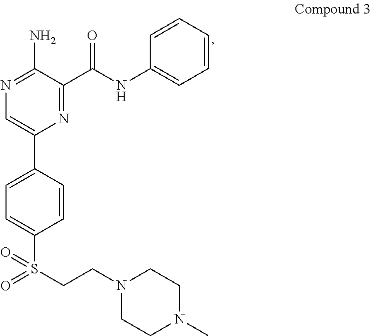 [SMILES: CN1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent.

In embodiments, the present disclosure provides a pharmaceutical combination comprising a liposomal composition comprising Compound 4:

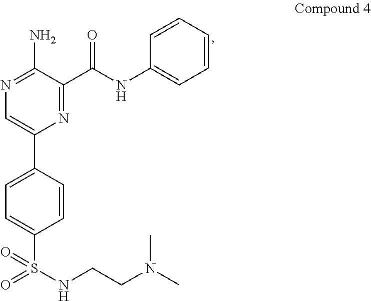 [SMILES: CN(C)CCNS(=O)(=O)c1ccc(-c2cnc(N)c(C(=O)Nc3ccccc3)n2)cc1]

or a pharmaceutically acceptable salt thereof and a chemotherapeutic agent.

In embodiments, the present disclosure provides a pharmaceutical combination comprising a liposomal composition comprising Compound 5:

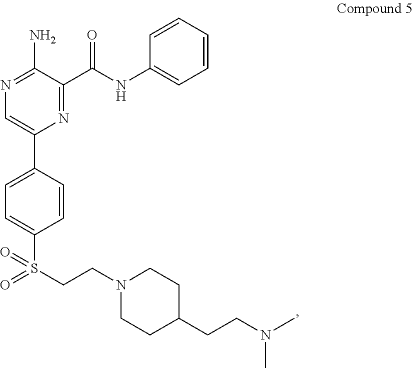 [SMILES: CN(C)CCC1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent.

In embodiments, the present disclosure provides a pharmaceutical combination comprising a liposomal composition comprising Compound 6:

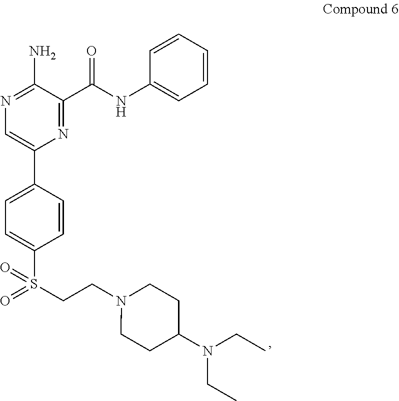 [SMILES: CCN(CC)C1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent.

In embodiments, the chemotherapeutic agent is irinotecan, topotecan, gemcitabine, cisplatin, or carboplatin.

In embodiments, the present disclosure provides a pharmaceutical combination comprising a liposomal composition comprising Compound 1:

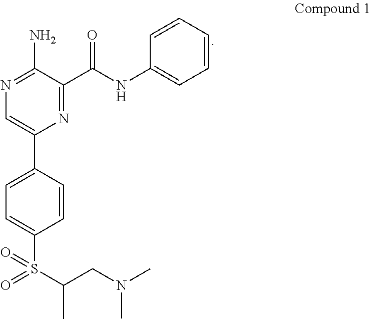 [SMILES: CC(CN(C)C)S(=O)(=O)c1ccc(-c2cnc(N)c(C(=O)Nc3ccccc3)n2)cc1]

or a pharmaceutically acceptable salt thereof and irinotecan or a pharmaceutically acceptable salt thereof.

In embodiments, the present disclosure provides a pharmaceutical combination comprising a liposomal composition comprising Compound 2:

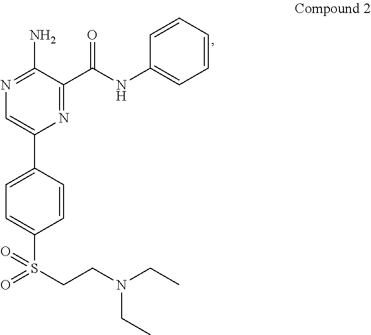 [SMILES: CCN(CC)CCS(=O)(=O)c1ccc(-c2cnc(N)c(C(=O)Nc3ccccc3)n2)cc1]

or a pharmaceutically acceptable salt thereof and irinotecan or a pharmaceutically acceptable salt thereof.

In embodiments, the present disclosure provides a pharmaceutical combination comprising a liposomal composition comprising Compound 3:

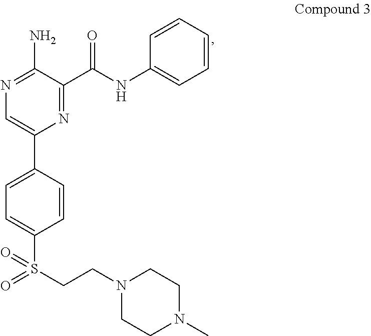 [SMILES: CN1CCN(CCS(=O)(=O)c2ccc(-c3cnc(N)c(C(=O)Nc4ccccc4)n3)cc2)CC1]

or a pharmaceutically acceptable salt thereof and irinotecan or a pharmaceutically acceptable salt thereof.

In embodiments, the present disclosure provides a pharmaceutical combination comprising a liposomal composition comprising Compound 4:

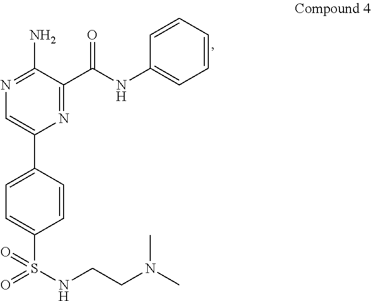 [SMILES: CN(C)CCNS(=O)(=O)c1ccc(-c2cnc(N)c(C(=O)Nc3ccccc3)n2)cc1]

or a pharmaceutically acceptable salt thereof and irinotecan or a pharmaceutically acceptable salt thereof.

In embodiments, the present disclosure provides a pharmaceutical combination comprising a liposomal composition comprising Compound 5:

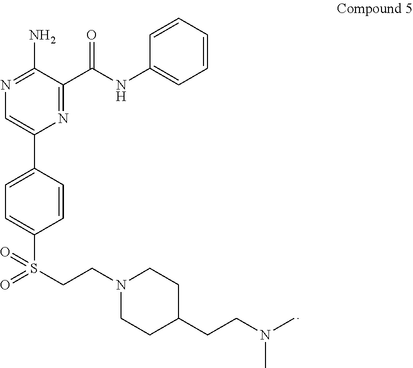 [SMILES: CN(C)CCC1CCN(CCS(=O)(=O)c2ccc(-c3cnc(N)c(C(=O)Nc4ccccc4)n3)cc2)CC1]

or a pharmaceutically acceptable salt thereof and irinotecan or a pharmaceutically acceptable salt thereof.

In embodiments, the present disclosure provides a pharmaceutical combination comprising a liposomal composition comprising Compound 6:

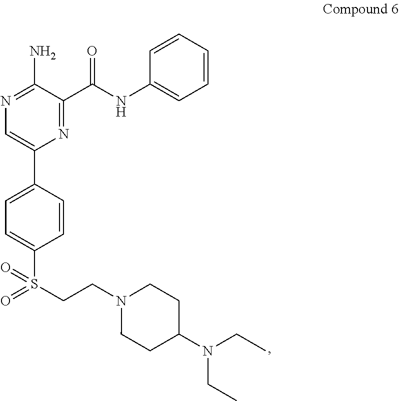 [SMILES: CCN(CC)C1CCN(CCS(=O)(=O)c2ccc(-c3cnc(N)c(C(=O)Nc4ccccc4)n3)cc2)CC1]

or a pharmaceutically acceptable salt thereof and irinotecan or a pharmaceutically acceptable salt thereof.

In embodiments, the present disclosure provides a pharmaceutical combination comprising a liposomal composition comprising Compound 1:

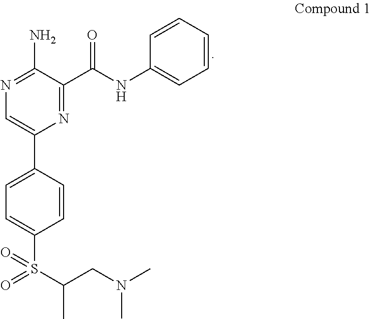 [SMILES: CC(CN(C)C)S(=O)(=O)c1ccc(-c2cnc(N)c(C(=O)Nc3ccccc3)n2)cc1]

or a pharmaceutically acceptable salt thereof and a chemotherapeutic agent selected from the group consisting of irinotecan, topotecan, gemcitabine, cisplatin, carboplatin or a pharmaceutically acceptable salt thereof.

In embodiments, the present disclosure provides a pharmaceutical combination comprising a liposomal composition comprising Compound 2:

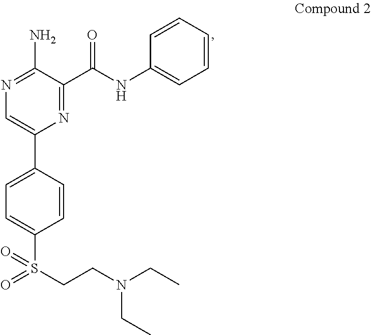 [SMILES: CCN(CC)CCS(=O)(=O)c1ccc(-c2cnc(N)c(C(=O)Nc3ccccc3)n2)cc1]

or a pharmaceutically acceptable salt thereof and a chemotherapeutic agent selected from the group consisting of irinotecan, topotecan, gemcitabine, cisplatin, carboplatin or a pharmaceutically acceptable salt thereof.

In embodiments, the present disclosure provides a pharmaceutical combination comprising a liposomal composition comprising Compound 3:

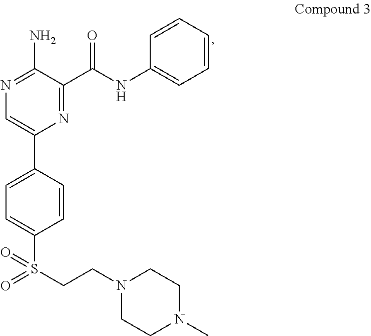 [SMILES: CN1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent selected from the group consisting of irinotecan, topotecan, gemcitabine, cisplatin, carboplatin or a pharmaceutically acceptable salt thereof.

In embodiments, the present disclosure provides a pharmaceutical combination comprising a liposomal composition comprising Compound 4:

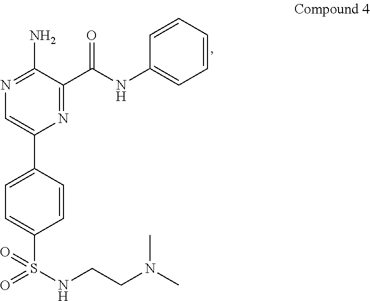 [SMILES: CN(C)CCNS(=O)(=O)c1ccc(-c2cnc(N)c(C(=O)Nc3ccccc3)n2)cc1]

or a pharmaceutically acceptable salt thereof and a chemotherapeutic agent selected from the group consisting of irinotecan, topotecan, gemcitabine, cisplatin, carboplatin or a pharmaceutically acceptable salt thereof.

In embodiments, the present disclosure provides a pharmaceutical combination comprising a liposomal composition comprising Compound 5:

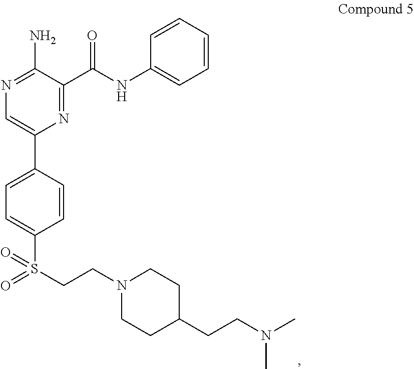 [SMILES: CN(C)CCC1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent selected from the group consisting of irinotecan, topotecan, gemcitabine, cisplatin, carboplatin or a pharmaceutically acceptable salt thereof.

In embodiments, the present disclosure provides a pharmaceutical combination comprising a liposomal composition comprising Compound 6:

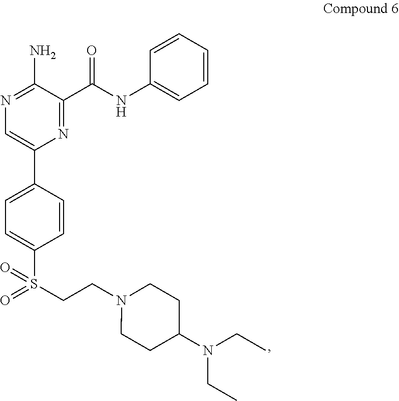 [SMILES: CCN(CC)C1CCN(CCS(=O)(=O)c2ccc(-c3cnc(N)c(C(=O)Nc4ccccc4)n3)cc2)CC1]

or a pharmaceutically acceptable salt thereof and a chemotherapeutic agent selected from the group consisting of irinotecan, topotecan, gemcitabine, cisplatin, carboplatin or a pharmaceutically acceptable salt thereof.

In some embodiments, the liposomal composition comprises a liposome-forming lipid, a cholesterol or a polymer-conjugated lipid. In some embodiments, the liposome-forming lipid comprises one or more phospholipids.

In some embodiments, exemplary liposomal membranes (the bilayer of phospholipids separating the internal aqueous medium from the external aqueous medium) comprises or may be formed from phospholipids, diglycerides, dialiphatic glycolipids, sphingomyelin, egg sphingomyelin (ESM), dihydrosphingomyelin (DHSM), glycosphingolipid, or cholesterol or derivates thereof, or combinations thereof. In some embodiments, the liposome-forming lipid or the liposome lipid comprise phospholipids, diglycerides, dialiphatic glycolipids, sphingomyelin, egg sphingomyelin (ESM), dihydrosphingomyelin (DHSM), glycosphingolipid, or cholesterol or derivates thereof, or combinations thereof.

In general, a variety of liposome lipid components can be used to make the liposomal composition as disclosed herein. Liposome lipid components can include, but are not limited to (a) uncharged lipid components, e.g., cholesterol, ceramide, diacylglycerol, acyl(poly ethers) or alkylpoly(ethers); (b)) neutral phospholipids, e.g., diacylphosphatidylcholines, sphingomyelins, and diacylphosphatidylethanolamines, (c) anionic lipids, e.g., diacylphosphatidylserine, diacylphosphatidylglycerol, diacylphosphatidate, cardiolipin, diacylphosphatidylinositol, diacylglycerolhemisuccinate, diaclyglycerolhemigluratate, cholesterylhemisuccinate, cholesterylhemiglutarate, and the like; (d) polymer-conjugated lipids, e.g., N-[methoxy-(poly(ethylene glycol)diacylphosphatidylethanolamine, poly(ethylene glycol)-diacylglycerol, poly(ethylene glycol)-ceramide; and (e) cationic lipids, e.g., 1,2, -diacyl-3-trimethylammonium-propane (DOTAP), dimethyldioctadecylammonium bromide (DDAB), and 1,2-diacyl-sn-glycero-3-ethylphosphocholine. Monoacyl-substituted derivatives of these lipids, as well as di- and monoalkyl-analogs can also be used.

Various liposome lipid components can be selected to fulfill, modify or impart one or more desired functions. Phospholipid can be used as principal vesicle-forming lipid. Inclusion of cholesterol can be useful for maintaining membrane rigidity and decreasing drug leakage. Polymer-conjugated lipids (such as PEG-lipid conjugates) can be used in the liposomal formulation to increase the lifetime of circulation via reducing liposome clearance by liver and spleen, or to improve the stability of liposomes against aggregation during storage, in the absence of circulation extending effect.

In some embodiments, the liposome lipid comprises a phospholipid, a cholesterol, and a PEG-lipid conjugate.

In some embodiments, the phospholipid is phosphatidic acid, phosphatidyl glycerol, phosphatidylcholine, phosphatidylethanolamine, phosphatidylinositol, phosphatidylserine, or mixtures thereof. In some embodiments, the phospholipid is 1,2-dipalmitoyl-sn-glycero-3-phosphocholine (DPPC), dimyristoyl-phosphatidylcholine (DMPC), hydrogenated soy phosphatidylcholine (HSPC), soy phosphatidylcholine (SPC), dimyristoylphosphatidylglycerol (DMPG), disrearoylphosphatidylglycerol (DSPG), 1-palmitoyl-2-oleoyl-sn-glycero-3-phosphocholine (POPC), 1,2-dioleoyl-sn-glycero-3-phosphocholine (DOPC), distearoyl phosphatidylcholine (DSPC), egg yolk phosphatidylcholine (EYPC) or hydrogenated egg yolk phosphatidylcholine (HEPC), sterol modified lipids (SML), cationic lipids and inverse-zwitterlipids. In some embodiments, the phospholipid is distearoylphosphatidylethanolamine (DSPE). In some embodiments, the phospholipid is 1,2-distearoyl-sn-glycero-3-phosphocholine (DSPC).

In some embodiments, phospholipid is a diacylphosphatidylcholine (PC). In some embodiments, PC is derived from natural sources. In some embodiments, PC is derived from synthetic sources.

In some embodiments, the PC is dipalmitoylphosphocholine (DPPC), dimyristoylphosphatidylcholine (DMPC), dimyristoylphosphatidylglycerol (DMPG), distearoylphosphatidylglycerol (DSPG), 1-palmitoyl-2-oleoyl-sn-glycero-3-phosphocholine (POPC), dioleoylphosphocholine (DOPC), distearoyl phosphatidylcholine (DSPC), or dilinoleoylphosphatidylcholine (DLPC). In some embodiments, the PC is soy phosphatidylcholine (SPC), hydrogenated soy phosphatidylcholine (HSPC), egg yolk phosphatidylcholine (EYPC) or hydrogenated egg yolk phosphatidylcholine (HEPC).

In some embodiments, the liposome comprises or is prepared with DSPC, cholesterol, or methoxy-poly(ethylene glycol)-1,2-distearoyl-sn-glyceryl (PEG-DSG), or a combination thereof. In some embodiments, the liposome comprises or is prepared with DSPC, cholesterol, and PEG-DSG.

In some embodiments, the liposome comprises or is prepared with DSPC, cholesterol, or 1,2-dimyristoyl-rac-glycero-3-methylpolyoxyethylene (PEG-DMG), or a combination thereof. In some embodiments, the liposome comprises or is prepared with DSPC, cholesterol, and PEG-DMG.

In some embodiment, the PEG-lipid conjugate is polyethylene glycol phosphatidylethanolamine, polyethylene glycol-diacylglycerol, or polyethylene glycol-ceramide derivative. In some embodiments, the PEG-lipid conjugate is PEG-DSG. In some embodiment, the PEG-lipid is PEG-DSG having a molecular weight from about 550 Daltons to about 5,000 Daltons. In some embodiments, PEG-DSG has a molecular weight of about 550 Daltons, about 600 Daltons, about 700 Daltons, about 800 Daltons, about 900 Daltons, about 1,000 Daltons, about 1,100 Daltons, about 1,200 Daltons, about 1,300 Daltons, about 1,400 Daltons, about 1,500 Daltons, about 1,600 Daltons, about 1,700 Daltons, about 1,800 Daltons, about 1,900 Daltons, about 2,000 Daltons, about 2,100 Daltons, about 2,200 Daltons, about 2,300 Daltons, about 2,400 Daltons, about 2,500 Daltons, about 2,600 Daltons, about 2,700 Daltons, about 2,800 Daltons, about 2,900 Daltons, about 3,000 Daltons, about 3,100 Daltons, about 3,200 Daltons, about 3,300 Daltons, about 3,400 Daltons, about 3,500 Daltons, about 3,600 Daltons, about 3,700 Daltons, about 3,800 Daltons, about 3,900 Daltons, about 4,000 Daltons, about 4,100 Daltons, about 4,200 Daltons, about 4,300 Daltons, about 4,400 Daltons, about 4,500 Daltons, about 4,600 Daltons, about 4,700 Daltons, about 4,800 Daltons, about 4,900 Daltons, or about 5,000 Daltons. In some embodiments, the PEG-DSG is PEG1000-DSG, PEG2000-DSG, or PEG3000-DSG. In some embodiments, the PEG-DSG is PEG2000-DSG.

In some embodiments, the PEG-lipid conjugate is PEG-DMG. In some embodiment, the PEG-lipid is PEG-DMG having a molecular weight from about 550 Daltons to about 5,000 Daltons. In some embodiments, PEG-DMG has a molecular weight of about 550 Daltons, about 600 Daltons, about 700 Daltons, about 800 Daltons, about 900 Daltons, about 1,000 Daltons, about 1,100 Daltons, about 1,200 Daltons, about 1,300 Daltons, about 1,400 Daltons, about 1,500 Daltons, about 1,600 Daltons, about 1,700 Daltons, about 1,800 Daltons, about 1,900 Daltons, about 2,000 Daltons, about 2,100 Daltons, about 2,200 Daltons, about 2,300 Daltons, about 2,400 Daltons, about 2,500 Daltons, about 2,600 Daltons, about 2,700 Daltons, about 2,800 Daltons, about 2,900 Daltons, about 3,000 Daltons, about 3,100 Daltons, about 3,200 Daltons, about 3,300 Daltons, about 3,400 Daltons, about 3,500 Daltons, about 3,600 Daltons, about 3,700 Daltons, about 3,800 Daltons, about 3,900 Daltons, about 4,000 Daltons, about 4,100 Daltons, about 4,200 Daltons, about 4,300 Daltons, about 4,400 Daltons, about 4,500 Daltons, about 4,600 Daltons, about 4,700 Daltons, about 4,800 Daltons, about 4,900 Daltons, or about 5,000 Daltons. In some embodiments, the PEG-DMG is PEG1000-DMG, PEG2000-DMG, or PEG3000-DMG. In some embodiments, the PEG-DSG is PEG2000-DMG.

In some embodiments, PEG in the PEG-lipid conjugate has a molecular weight from about 250 to about 20,000. In some embodiments, PEG in the PEG-lipid conjugate has a molecular weight from about 500 to about 5,000. In some embodiments, PEG in the PEG-lipid conjugate has a molecular weight of about 1000, about 2000, or about 3000.

In some embodiments, the liposome lipid comprises lecithin. In some embodiments, lecithin is a natural lecithin, a hydrogenated natural lecithin, a synthetic lecithin, 1,2-distearoyl-lecithin, dipalmitoyl lecithin, dimyristoyl lecithin, dioleolyl lecithin, 1-stearoyl-2-oleoyl lecithin, or 1-palmitoyl-2-oleoyl lecithin.

In some embodiments, the liposome lipid comprises phytosterol. In some embodiments, the liposome lipid comprises beta-sitosterol.

In some embodiments, the liposome lipid comprises dioleoylphosphatidylethanolamine (DOPE).

In some embodiments, the liposome comprises about 50 mol % to about 70 mol % of a phospholipid. In some embodiments, the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of a phospholipid.

In some embodiments, the liposome comprises about 50 mol % to about 70 mol % of DSPC. In some embodiments, the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

In some embodiments, the liposome comprises about 30 mol % to about 50 mol % of cholesterol. In some embodiments, the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

In some embodiments, the liposome comprises about 0.1 mol % to about 5 mol % of a PEG-lipid conjugate. In some embodiments, the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of a PEG-lipid conjugate.

In some embodiments, the liposome comprises about 0.1 mol % to about 2.5 mol % of a PEG-lipid conjugate.

In some embodiments, the liposome comprises about 0.1 mol % to about 2 mol % of a PEG-lipid conjugate.

In some embodiments, the liposome comprises about 0.1 mol % to about 0.5 mol % of a PEG-lipid conjugate.

In some embodiments, the molar ratio of phospholipid:cholesterol:PEG-lipid conjugate of the liposome is about 27:20:1, about 26:20:1, or about 25:20:1.

In some embodiments, the liposome comprises phospholipid:cholesterol:PEG-lipid conjugate in molar ratio of between about 59.9:40:0.1 and about 58:40:2.

In some embodiments, the liposome comprises phospholipid:cholesterol:PEG-lipid conjugate in molar ratio of between about 59.9:40:0.1 and about 59.75:40:25.

In some embodiments, the liposome comprises phospholipid:cholesterol:PEG-lipid conjugate in molar ratio of between about 59.9:40:0.1 and about 58:40:2

In some embodiments, the molar ratio of phospholipid:cholesterol:PEG-lipid conjugate of the liposome is about 58:40:2 or about 59.75:40:0.25.

In some embodiments, the molar ratio of phospholipid:cholesterol:PEG-lipid conjugate of the liposome is about 59.75:40:0.25.

In some embodiments, the molar ratio of phospholipid:cholesterol:PEG-lipid conjugate of the liposome is between about 3:2:0.05 and about 3:2:0.10.

In some embodiments, the liposome comprises about 0.1 mol % to about 5 mol % of PEG-DSG. In some embodiments, the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG. In some embodiments, the liposome comprises less than 1 mol % PEG-DSG. In some embodiments, the liposome comprises less than 0.5 mol % PEG-DSG. In some embodiments, the liposome comprises less than 0.3 mol % PEG-DSG.

In some embodiments, the liposome comprises about 0.1 mol % to about 2.5 mol % PEG-DSG.

In some embodiments, the liposome comprises about 0.1 mol % to about 2 mol % PEG-DSG.

In some embodiments, the liposome comprises about 0.1 mol % to about 0.5 mol % PEG-DSG.

In some embodiments, the amount of PEG-lipid conjugate in the liposome is kept at its minimal amount possible to avoid or minimize adverse reactions to subjects. In some embodiments, the amount of PEG-lipid conjugate in the liposome does not cause or induce accelerated blood clearance phenomenon of the liposome.

In some embodiments, the molar ratio of DSPC: cholesterol of the liposome is in the range of about 1:1 to about 2:1. In some embodiments, the molar ratio of DSPC: cholesterol of the liposome is in the range of about 14:10 to about 15:10. In some embodiments, the molar ratio of DSPC: cholesterol of the liposome is about 14:10, about 13:10, or about 12:10.

In some embodiments, the molar ratio of cholesterol: PEG-DSG of the liposome is in the range of about 10:1 to about 100:1. In some embodiments, the molar ratio of cholesterol: PEG-DSG of the liposome is about 20:1. In some embodiments, the molar ratio of cholesterol: PEG-DSG of the liposome is about 400:2, about 400:2.5, or about 400:3.

In some embodiments, the molar ratio of DSPC: cholesterol: PEG-DSG of the liposome is about 27:20:1, about 26:20:1, or about 25:20:1.

In some embodiments, the liposome comprises DSPC, cholesterol, and PEG-DSG in molar ratio of between about 59.9:40:0.1 and about 58:40:2.

In some embodiments, the liposome comprises DSPC, cholesterol, and PEG-DSG in molar ratio of between about 59.9:40:0.1 and about 59.75:40:25.

In some embodiments, the liposome comprises DSPC, cholesterol, and PEG-DSG in molar ratio of between about 59.9:40:0.1 and about 58:40:2

In some embodiments, the molar ratio of DSPC: cholesterol: PEG-DSG of the liposome is about 58:40:2 or about 59.75:40:0.25.

In some embodiments, the molar ratio of DSPC: cholesterol: PEG-DSG of the liposome is about 59.75:40:0.25.

In some embodiments, the molar ratio of DSPC: cholesterol: PEG-DSG of the liposome is between about 3:2:0.05 and about 3:2:0.10.

In some embodiments of the liposomal composition, the therapeutically active agent: liposome lipids molar ratio is in the range of about 0.05 to about 2.0. In some embodiments, the therapeutically active agent: liposome lipids molar ratio is in the range of about 0.1 to about 1.0. In some embodiments, the therapeutically active agent: liposome lipids molar ratio is about 0.2, about 0.3, about 0.4, about 0.5, about 0.6, about 0.7, about 0.8, about 0.9 or about 1.0. In some embodiments, the therapeutically active agent: liposome lipids molar ratio is about 0.3.

In some embodiments of the liposomal composition, the liposome encapsulation efficiency is greater than 80%. In some embodiments, the liposome encapsulation efficiency is greater than 90%. In some embodiments, the liposome encapsulation efficiency is greater than 95%. In some embodiments, the liposome encapsulation efficiency is about 90%, about 91%, about 92%, about 93%, about 94%, about 95%, about 96%, about 97%, about 98%, about 99%, or about 100%.

In some embodiments of the liposomal composition, an average size (mean diameter) of the liposomes is in the range of about 80 nm to about 150 nm. In some embodiments of the liposomal composition, an average size (mean diameter) of the liposomes is in the range of about 80 nm to about 120 nm. In some embodiments, the average size (mean diameter) of the liposomes is about 90 nm, about 95 nm, about 100 nm, about 105 nm, about 110 nm, about 115 nm, or about 120 nm.

In some embodiments, the liposomal composition of the invention provides high cell uptake of the compound of formula (I) or a pharmaceutically acceptable salt thereof.

In some embodiments, the liposomal composition of the invention provides lowered IC₅₀ compared to the same ATRi in a non-liposome composition.

In some embodiments, the liposomal composition comprises a compound of formula (I) or a pharmaceutically acceptable salt thereof, liposome lipids, and a pharmaceutically acceptable carrier or excipient.

In some embodiments, a pharmaceutically acceptable carrier is a buffer, normal saline, isotonic dextrose, isotonic sucrose, Ringer's solution, or Hanks' solution. A buffer can comprise histidine, glycine, hydroxyethylpiperazine-ethylsulfonate (HEPES), morpholipo-ethylsulfonate (MES), succinate, tartrate, or citrate as buffer substance.

Methods of preparing and characterizing pharmaceutical liposomal compositions are known in the field (see, e.g., Lasic D. Liposomes: From physics to applications, Elsevier, Amsterdam 1993; G. Gregoriadis (ed.), Liposome Technology, 3rd edition, vol. 1-3, CRC Press, Boca Raton, 2006; Hong et al., U.S. Pat. No. 8,147,867, incorporated by reference herein in their entirety for all purposes). For liposome loading techniques, see also, U.S. Pat. Nos. 7,744,921, 7,238,367, 8,329,213, 8,703,181, 10,722,467 and 10,004,759, each of which are herein incorporated by reference in its entirety for all purposes. For various methods of preparing liposomes and encapsulation of therapeutic agents: see, for example, U.S. Pat. Nos. 3,932,657, 4,235,871. 4,311,712, 4,743,449, 4,452,747, 4,830,858, 4,921,757, and 5,013,556, each of which are herein incorporated by reference in its entirety for all purposes.

In some embodiments, the liposomal composition is prepared by an active loading technique. In some embodiments, the liposomal composition is prepared by a passive loading technique.

In some embodiments, preparation of the liposomal composition comprises a step of preparing a blank liposome. In some embodiments, the blank liposome is prepared using a thin film technique.

In some embodiments, preparation of the liposomal composition comprises a step of hydrating the blank liposome with a buffer. In some embodiment, the hydrating step is performed with a TEA₈SOS buffer. In some embodiments, the hydrating step is performed with a 1.1N TEA₈SOS buffer.

In some embodiments, preparation of the liposomal composition comprises a step of high-pressure extrusion following the hydrating step.

In some embodiments, preparation of the liposomal composition comprises a step of exchanging an external liposomal buffer. In some embodiments, the external liposomal buffer is exchanged for dextrose buffer. In some embodiments, the external liposomal buffer is exchanged for 18% dextrose buffer.

In some embodiments, preparation of the liposomal composition comprises a step of further exchanging the external liposomal buffer. In some embodiments, the external liposomal buffer is exchanged for HEPES buffered saline (pH 7.4).

In some embodiments, preparation of the liposomal composition comprises a step of actively loading the compound of formula (I) or a pharmaceutically acceptable salt thereof into the blank liposome. In some embodiments, the active loading is done on the blank liposome which was hydrated, extruded, and the external liposomal buffer exchanged.

In some embodiments, the compound of formula (I) or a pharmaceutically acceptable salt thereof is dissolved in a suitable solvent prior to active loading.

In some embodiments, preparation of the liposomal composition comprises a step of heating the liposome past the phase transition temperature after the compound of formula (I) or a pharmaceutically acceptable salt thereof is added. In some embodiments, the heating is performed while stirring. In some embodiments, heating is performed for about 1 hour.

In some embodiments, the loaded liposomes are cooled and purified. In some embodiments, purification comprises exchanging the external liposomal buffer. In some embodiments, purification comprises purifying the external liposomal buffer to eliminate any un-loaded compound of formula (I) or a pharmaceutically acceptable salt thereof.

In some embodiments, the purified loaded liposomes are concentrated to the desired concentration. In some embodiments, the concentrating step is performed using the tangential flow.

In some embodiments, pharmaceutical compositions can comprise nanoparticles. The formation of nanoparticles has been achieved by various methods. Nanoparticles can be made by precipitating a molecule in a water-miscible solvent, and then drying and pulverizing the precipitate to form nanoparticles. (U.S. Pat. No. 4,726,955). Similar techniques for preparing nanoparticles for pharmaceutical preparations include wet grinding or milling. Other methods include mixing low concentrations of polymers dissolved in a water-miscible solution with an aqueous phase to alter the local charge of the solvent and form a precipitate through conventional mixing techniques. (U.S. Pat. No. 5,766,635). Other methods include the mixing of copolymers in organic solution with an aqueous phase containing a colloid protective agent or a surfactant for reducing surface tension. Other methods of incorporating additive therapeutic agents into nanoparticles for drug delivery require that nanoparticles be treated with a liposome or surfactant before drug administration (U.S. Pat. No. 6,117,454). Nanoparticles can also be made by flash nanoprecipitation (U.S. Pat. No. 8,137,699).

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient.

### Therapeutic Use

The present disclosure also relates to method for using the composition of the invention for treating various diseases and conditions. In embodiments, the disease or a condition is implicated by one or more abnormal ATR activities.

The present disclosure provides method for treating or preventing cancer comprising administering to a subject in need thereof an effective amount of the composition of the invention.

In some embodiments, the disease or the condition is cancer. In some embodiments, the disease or the condition is a solid tumor. In embodiments, the disease or the condition is selected from Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain cancer; brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancers.

In embodiments, the present disclosure provides a method of treating cancer in a mammal in need thereof, comprising administering a liposomal composition comprising:
(i) a compound of formula (I) or a pharmaceutically acceptable salt thereof; and
(ii) a liposome comprising a phospholipid and a cholesterol,
with the proviso that the cancer is not lung cancer or breast cancer.

In embodiments, the present disclosure provides a method of treating cancer in a mammal in need thereof, comprising administering a liposomal composition comprising:
(i) a compound of formula (I) or a pharmaceutically acceptable salt thereof; and
(ii) a liposome comprising 1,2-distearoyl-sn-glycero-3-phosphocholine (DSPC) and a cholesterol.

In some embodiments, the cancer is lung cancer, brain cancer, colorectal cancer, ovarian cancer, breast cancer, gastric cancer, or endometrium cancer.

In some embodiments, the cancer is lung cancer, brain cancer, colorectal cancer, ovarian cancer, or breast cancer. In some embodiments, the lung cancer is small cell lung cancer or non-small cell lung cancer. In some embodiments, the breast cancer is triple negative breast cancer.

In some embodiments, the cancer is gastric cancer. In some embodiments, the cancer is gastroesophageal adenocarcinoma.

In some embodiments, the cancer is ovarian cancer, colorectal cancer, or lung cancer.

In some embodiments, the cancer is ovarian cancer, colorectal cancer, or small cell lung cancer.

In embodiments, the cancer is ovarian cancer, pancreatic cancer, prostate cancer, brain cancer, melanoma, gastric cancer, or endometrium cancer.

In some embodiments, the cancer is ovarian cancer or colorectal cancer.

In some embodiments, the cancer is ovarian cancer. In embodiments, the ovarian cancer is high-grade serous ovarian carcinoma (HGSOC).

In some embodiments, the cancer is colorectal cancer.

In some embodiments, the cancer is lung cancer.

In some embodiments, the cancer is small cell lung cancer.

In some embodiments, the cancer is non-small cell lung cancer.

In embodiments, the methods of the invention comprise detecting one or more of TP53, BRAF, SLFN, BRCA1, and ERBB2 in the subject to be treated with any of the methods disclosed herein. In embodiments, the subject is determined to have TP53 mutant cancer. In embodiments, the subject is determined to have TP53^{spl} cancer. In embodiments, the subject is determined to have BRAF mutant cancer e.g., BRAF^{V600E}. In embodiments, the subject is determined to have SLFN111^{lo} cancer. In embodiments, the subject is determined to have BRCA1^{methylated} cancer. In embodiments, the subject is determined to have ERBB2G^{776V} mutant cancer.

In one particular embodiment, the cancer is not one or more of the following cancers: cervical cancer, colorectal cancer, lung cancer, triple negative breast cancer, or osteosarcoma, In one embodiment, the cancer is not breast cancer or lung cancer. In one embodiment the cancer is not breast cancer. In one embodiment the cancer is not lung cancer. In one embodiment, the cancer is not lung squamous cell carcinoma, small cell lung cancer, or non-small cell lung cancer.

The present disclosure also relates to methods for treating a disease or a condition which is implicated by one or more abnormal ATR activities in a subject in need thereof, comprising administering a liposomal composition of an ATRi of the invention, wherein the method provides reduced toxicity when compared to administering the ATRi in a non-liposomal composition at the same dose. In some embodiments, the toxicity is neurological toxicity. In some embodiments, the toxicity is hematological toxicity. In some embodiments, the toxicity is long-term hematological toxicity. In some embodiments, the disease or the condition which is implicated by one or more abnormal ATR activities is cancer.

In some embodiments, the effective amount of the combination of the invention is an amount that provides therapeutic synergy between the compound of formula (I) and the additional therapeutically active agent.

In some embodiments, the methods of the invention provide therapeutic synergy between the compound of formula (I) or a pharmaceutically acceptable salt thereof, and an additional pharmaceutically active agent for example an anti-cancer agent anti-cancer agent, such as irinotecan. In some embodiments, the methods of the invention provide greater tumor growth inhibition when compared to the respective monotherapies e.g., a compound of formula (I) or the additional pharmaceutically active agent alone. In some embodiments, the methods of the invention provide at least 5% greater tumor growth inhibition with the combination therapy when compared to the respective monotherapies. In some embodiments, the methods of the invention provide at least 10% greater tumor growth inhibition with the combination therapy when compared to the respective monotherapies. In some embodiments, the methods of the invention provide at least 15% greater tumor growth inhibition with the combination therapy when compared to the respective monotherapies. In some embodiments, the methods of the invention provide at least 20% greater tumor growth inhibition with the combination therapy when compared to the respective monotherapies. In some embodiments, the methods of the invention provide at least 30% greater tumor growth inhibition with the combination therapy when compared to the respective monotherapies.

In some embodiments, the methods of the invention provide greater tumor growth inhibition when compared administering the compound of formula (I) or a pharmaceutically acceptable salt thereof in a non-liposome formulation. In some embodiments, the methods of the invention provide therapeutic synergy between the compound of formula (I) and irinotecan. In some embodiments, the methods of the invention provide greater tumor growth inhibition when compared administering irinotecan alone (monotherapy) in a liposome-free formulation. In some embodiments, the methods of the invention provide at least 5% greater tumor growth inhibition when compared to monotherapy with liposome-free irinotecan. In some embodiments, the methods of the invention provide at least 10% greater tumor growth inhibition when compared to monotherapy with liposome-free irinotecan. In some embodiments, the methods of the invention provide at least 15% greater tumor growth inhibition when compared to monotherapy with liposome-free irinotecan. In some embodiments, the methods of the invention provide at least 20% greater tumor growth inhibition when compared to monotherapy with liposome-free irinotecan.

In some embodiments, the methods of the invention results in greater plasm concentration of the compound of formula (I) or a pharmaceutically acceptable salt thereof when compared to a method where the compound of formula (I) or a pharmaceutically acceptable salt thereof is administered in a liposome-free composition at the same dose.

In some embodiments, the methods of the invention results in greater tumor tissue concentration of the compound of formula (I) or a pharmaceutically acceptable salt thereof when compared to a method where the compound of formula (I) or a pharmaceutically acceptable salt thereof is administered in a liposome-free composition at the same dose.

In some embodiments, the methods of the invention results in improved toxicity profile compared to a method where the compound of formula (I) or a pharmaceutically acceptable salt thereof is administered in a liposome-free composition at the same dose.

In some embodiments, the methods of the invention results in improved toxicity profile when compared to standard of care of the disease to be treated.

In some embodiments, the methods of the invention result in improved erythroid toxicity when compared to standard of care of the disease to be treated.

In some embodiments, the methods of the invention do not cause acute neurological toxicity. In some embodiments, the methods of the invention do not cause severe acute neurological toxicity. In some embodiments, the methods of the invention do not cause hematological toxicity. In some embodiments, the methods of the invention do not cause long-term hematological toxicity. In some embodiments, the methods of the invention do not cause erythroid toxicity.

In some embodiments, the methods of the invention do not cause the subject to have pancytopenia. In some embodiments, the methods of the invention protect the subject from pancytopenia. In some embodiments, the methods of the invention do not cause significant reduction in white blood cell count. In some embodiments, the methods of the invention do not cause significant reduction in reticulocytes.

In some embodiments, the methods of the invention do not cause the subject to have mild, moderate or severe anemia. In some embodiments, the methods of the invention do not cause the subject to have mild, moderate or severe reticulocytopenia. In some embodiments, the methods of the invention do not cause the subject to have an absolute reticulocyte count of less than about 10,000/μL, less than about 20,000/μL, less than about 30,000/μL, less than about 40,000/μL, less than about 50,000/μL, or less than about 60,000/μL. The reticulocyte count can be expressed either as a percentage of all red blood cells (RBCs), the absolute reticulocyte count, the corrected reticulocyte count, the reticulocyte production index (RPI) or as the immature reticulocyte fraction (IRF).

In some embodiments, the methods of the invention do not cause the subject to have mild, moderate or severe neutropenia. In some embodiments, the methods of the invention do not cause the subject to have a neutrophil count of less than about 1500/μL, less than about 1000/μL, or less than about 500/μL. In some embodiments, the methods of the invention do not cause the subject to have a neutrophil count of less than a range of about 1000-1500/μL, less than a range of about 500-1000/μL, or less than about 500/μL.

In some embodiments, the methods of the invention do not cause the subject to have mild, moderate or severe leukopenia. In some embodiments, the methods of the invention do not cause the subject to have grade 1, grade 2, grade 3 or grade 4 leukopenia. In some embodiments, the methods of the invention do not cause the subject to have a total leukocyte count of less than about 5,000-17,000/mm³. In some embodiments, the methods of the invention do not cause the subject to have a total leukocyte count of less than a range of about 5,000-11,000/μL. In some embodiments, the methods of the invention do not cause the subject to have a total leukocyte count of less than about 5,000/μL

In some embodiments, the liposomal composition comprises a compound of Table A, or a pharmaceutically acceptable salt thereof.

In some embodiments of the methods of the invention, the method further comprises administering an additional therapeutically active agent. In some embodiments, the method further comprises administering an additional therapeutically active agent in conjunction with the compound of formula (I) or a pharmaceutically acceptable salt thereof.

In some embodiments, the additional pharmaceutically active agent is irinotecan.

In some embodiments, the irinotecan is administered in a non-liposomal composition.

In some embodiments of the methods of the invention where the compound of formula (I) or a pharmaceutically acceptable salt thereof and the additional therapeutically active agent is administered in the same or different liposomal composition. In some embodiments, the compound of formula (I) or a pharmaceutically acceptable salt thereof and the additional therapeutically active agent is administered in the same liposomal composition (i.e., encapsulated in same liposome). In some embodiments, the compound of formula (I) or a pharmaceutically acceptable salt thereof and the additional therapeutically active agent is administered in different liposomal compositions. In some embodiments, the compound of formula (I) or a pharmaceutically acceptable salt thereof is administered in a liposomal composition and the additional therapeutically active agent is administered in liposome-free composition. In some embodiment, administration of the compound of formula (I) or a pharmaceutically acceptable salt thereof and the additional therapeutically active agent is performed simultaneously, sequentially, or continuously, through the same or different routes. In some embodiment, administration of the compound of formula (I) or a pharmaceutically acceptable salt thereof and the additional therapeutically active agent is done on a separate dosing interval.

In some embodiments of the methods of the invention, the compound of formula (I) or a pharmaceutically acceptable salt thereof is administered at a dose in the range of about 1 mg/m² to about 500 mg/m². In one embodiment, the compound of formula (I) or a pharmaceutically acceptable salt thereof is administered at a dose in the range of about 20 mg/m² to about 400 mg/m². In some embodiments, the compound of formula (I) or a pharmaceutically acceptable salt thereof is administered at a dose in the range of about 30 mg/m² to about 200 mg/m².

In some embodiments of the methods of the invention, the additional therapeutically active agent is administered at a dose in the range of about 1 mg/m² to about 1000 mg/m². In one embodiment, the additional therapeutically active agent is administered at a dose in the range of about 50 mg/m² to about 500 mg/m².

In some embodiments of the methods of the invention, the compound of formula (I) or a pharmaceutically acceptable salt thereof is administered once a day, twice a day, or three times a day. In some embodiments of the methods of the invention, the compound of formula (I) or a pharmaceutically acceptable salt thereof is administered once every 2 days, once every 3 days, once every 4 days, once every 5 days, once every 6 days, or once every 7 days. In some embodiments of the methods of the invention, the compound of formula (I) or a pharmaceutically acceptable salt thereof is administered once a week, once every two weeks, once every three weeks, or once every four weeks.

In some embodiments of the methods of the invention, the compound of formula (I) or a pharmaceutically acceptable salt thereof is administered once a week (QW).

In some embodiments of the methods of the invention, the additional therapeutically active agent is administered once a day, twice a day, or three times a day. In some embodiments of the methods of the invention, the additional therapeutically active agent is administered once every 2 days, once every 3 days, once every 4 days, once every 5 days, once every 6 days, or once every 7 days. In some embodiments of the methods of the invention, the additional therapeutically active agent is administered once a week, once every two weeks, once every three weeks, or once every four weeks.

### EXAMPLES

The disclosure now being generally described, it will be more readily understood by reference to the following examples which are included merely for purposes of illustration of certain aspects and embodiments of the present invention, and are not intended to limit the invention.

### Example 1. Preparation of Liposomal Formulation

Blank liposomes, including those containing DSPC: cholesterol: PEG-DSG at relative molar ratios according to Table 1, were formulated using the thin film technique followed by 0.5-1.1N TEA₈SOS buffer (“TEA-SOS”) hydration and high-pressure liquid extrusion. Some liposomes used ammonium sulfate (“AS”) buffer at either low (“LAS”; 250 mM) or high (“HAS”; 500 mM) concentration. Prior to immediate drug loading, the external liposomal buffer was exchanged for 18% dextrose buffer.

The compounds of formula (I)(including Compounds A-C, which are compounds from Table A) were actively loaded into the blank liposomes. The Compounds were dissolved in DMSO, and actively loaded into the liposomes in the presence of citrate buffer (pH range of 4-6), to a final concentration of about 2% v/v DMSO. Drug added liposomes were heated past the phase transition temperature (i.e., 65° C.) and stirred for 1 hour. Resultant drug loaded liposomes were then cooled on ice for 5 minutes before further purification of external, un-loaded drug (in external liposomal medium) using tangential flow against HEPES buffered saline. Liposomes were then concentrated to the desired drug concentration using tangential flow.

Drug loading was assessed using HPLC-UV. ATR inhibitors of formula (I) were extracted from intact liposomes for HPLC-UV.

Lipid analysis was conducted by ELSD.

Prepared liposomal Formulations are listed in Table 1.

TABLE 1
Liposomal Formulations

| No. | Liposomal Lipids | | | Ratio | Loading Aid | Loading Method |
|---|---|---|---|---|---|---|
| A | DSPC | Cholesterol | PEG-DSG | 58:39:3 | TEA-SOS | Low pH |
| B | DSPC | Cholesterol | PEG-DSG | 58:40:2 | TEA-SOS | DMSO |
| C | ESM | Cholesterol | PEG-DSG | 53:45:2 | TEA-SOS | Low pH |
| D | DSPC | Cholesterol | PEG-DSG | 58:40:2 | TEA-SOS | Low pH |
| E | DSPC | Cholesterol | PEG-DSG | 58:40:2 | TEA-SOS | DMSO/Citrate |
| F | DSPC | Cholesterol | PEG-DSG | 58:40:2 | TEA-SOS | Neutral pH |
| G | DSPC | Cholesterol | PEG-DSG | 59.75:40:0.25 | TEA-SOS | DMSO/Citrate |
| H | DSPC | Cholesterol | PEG-DMG | 59.75:40:0.25 | TEA-SOS | DMSO/Citrate |
| I | DSPC | Cholesterol/B-sito | PEG-DSG | 59.75:10:30:0.25 | TEA-SOS | DMSO/Citrate |
| J | DSPC | Cholesterol/DOPE | PEG-DSG | 49.75:10:40:0.25 | TEA-SOS | DMSO/Citrate |
| K | DSPC | Cholesterol | PEG-DSG | 59.75:40:0.25 | LAS | DMSO/Citrate |

B-sito = beta-sitosterol

### Example 2. In Vitro Study of the Liposomal Formulation of ATR Inhibitors

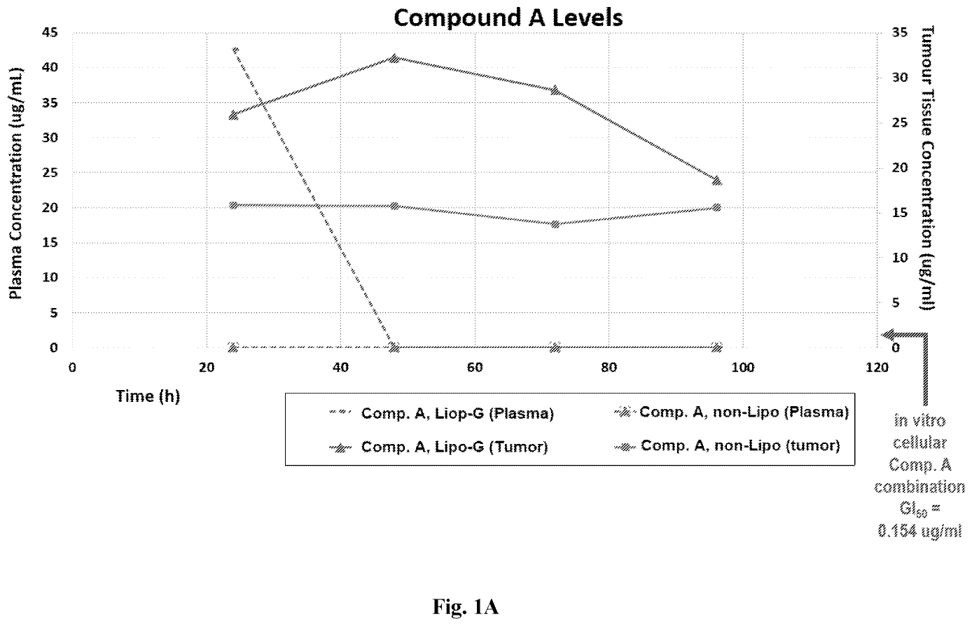

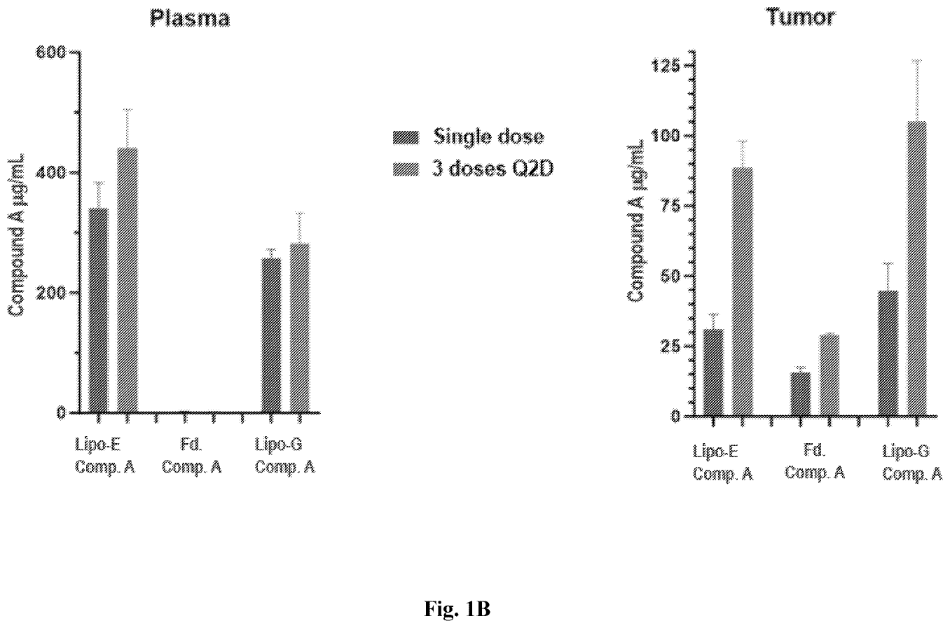

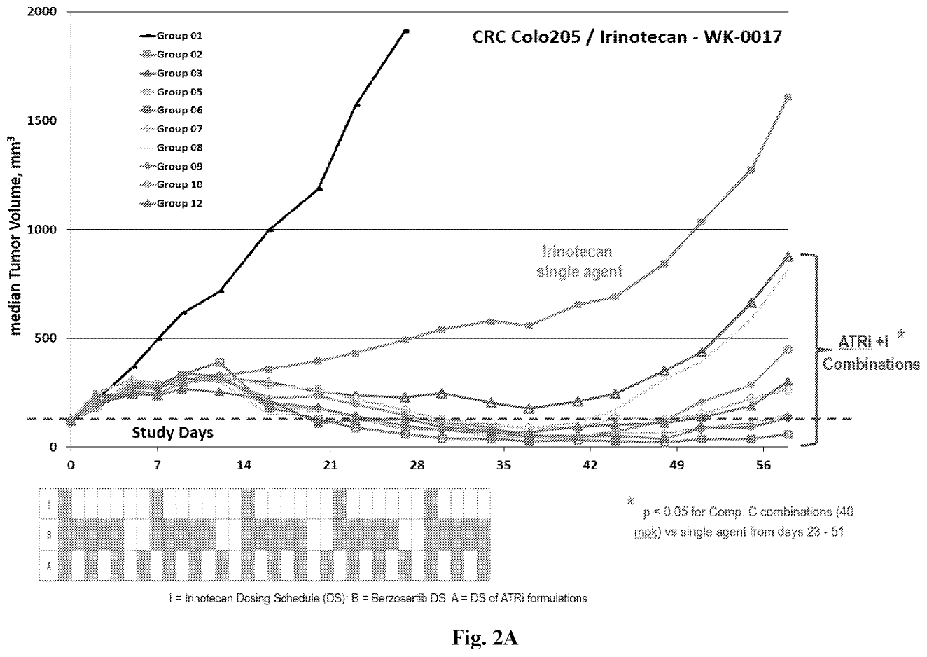

Pharmacokinetic studies were used to measure in vivo activity. The pharmacokinetic profile of liposomal Formulation G from Example 1, “Lipo-G” or liposomal Formulation E from example 1, “Lipo-E” and non-liposomal formulations of ATR inhibitors of formula (I) were assessed at various dose changes in healthy, 6-8-week-old athymic nude mice in CRC-Colo205 tumor model. ATR inhibitors were administered once to mice via IV at 40 mg/kg. Blood samples were collected by cardiac puncture at timepoints of 24 hours, 48 hours, 72 hours, and 96 hours. FIG. 1A show concentration of ATRi (Compound A) in plasma and in tumor tissue over time. FIG. 1B shows the concentration of ATRi (Compound A) in Lipo-G or Lipo-E in plasma and tumor compared to free Compound A after a single dose or three doses (Q2D). As shown in FIG. 1B liposomal Compound A formulations show higher tumor accumulation compared to free Compound A. Approximately 3-fold higher ATR tumor levels were observed after 3 doses than a single dose of liposomal Compound A formulations.

Sustained systemic exposure of Compound A in plasma was observed with liposomal formulation where t1/2 of about 20 hours, while Compound A from the non-liposomal formulation was undetectable at the same time (below limit of detection ~2 μg/mL).

Peak accumulation of Compound A in tumor tissues was at 1-2 days for the liposomal composition with slow rate of decline. Total Compound A levels in the tumor provide >100-fold coverage over in vitro GI₅₀ concentration (expected efficacious dose). In vitro GI₅₀ for Compound A was calculated in a genotoxic combination (gemcitabine held constant concentration at its own single agent GI50/75, 4d) in PDAC(PANC1 line) at 2-4 days.

Non-liposomal Compound A (free drug) demonstrates triple digit nanomolar GI50 potencies across a panel of solid tumor cancer cell lines in vitro. Similar level of potency of Compound A from liposomal Compound A (liposomal drug) in combination with irinotecan is expected in CRC as in PDAC in vitro.

Half-life for free drug and liposomal drug were 15 minutes and 10 hours, respectively. The AUCs for free drug and liposomal drug were 50 and 1255 μg/(mLhr), respectively.

### Example 3. Efficacy of Liposomal ATR Inhibitors

### Liposomal ATRi+Irinotecan Vs Irinotecan Alone

Tumor efficacy studies were used to compare the efficacy of ATR inhibitors (Compounds A and C) in liposomal formulations in combination with irinotecan against irinotecan as a single agent in CRC Colo205 model.

Subcutaneous tumor models were initiated on 6-8 week old, female athymic nude mice. A cell suspension containing 5e6/100 μL CRC Colo205 cells in 1:1 PBS: Matrigel was subcutaneously injected into the hind flank of NSG mice. Tumors were measured three times a week by digital caliper and reported as volume, calculated as V=π×1×w∧2 wherein 1 and w denote the longest and shortest tumor diameter respectively. Engrafted mice were then randomized into cohorts for treatment at an average tumor size of 80 mm³.

Mice were organized into appropriate groups consisting of control (empty vehicle) and treatment groups including free irinotecan, irinotecan in combination with liposomal Compound A or liposomal Compound C, or irinotecan and berzosertib. Mice were administered free drug orally, and injected intravenously for liposomal compounds, with the required volume of sample to administer the prescribed dose to the animals based on individual mouse weights on dosing days. Animals were weighed and monitored for survival and in-life observations were collected at the time of weight measurement. Mice were monitored individually to endpoint which was either an individual tumor weight of 2000 mm³ or other defined risk factors, whichever came first.

Animal study groups are disclosed in Table 2 (n=8 per group). Irinotecan was administered at 50 mg/kg via IP in a liposomal-free composition, once a week. Liposomal ATR inhibitors were administered at either 20 mg/kg (Group7) or 40 mg/kg (Groups 3, 5, 6, 8-10) via IV every other day. Berzosertib, clinical benchmark, was administered 5 consecutive days with 2 days off schedule. Last dose of any therapeutically active agent was administered on day 33.

TABLE 2
Study Details

| | | | | ATRi Lipo |
|---|---|---|---|---|
| | | | Dose of ATRi | Formulation |
| Group | | | or | from |
| No. | Treatment | Dose of I | Berzosertib | Example 1 |
| 1 | Vehicle | none | none | NA |
| 2 | Irinotecan | 50 mg/kg IP | none | NA |
| | (“I”) | | | |
| 3 | I + Comp. C | 50 mg/kg IP | 40 mg/kg IV | K |
| 5 | I + Comp. A | 50 mg/kg IP | 40 mg/kg IV | E |
| 6 | I + Comp. A | 50 mg/kg IP | 40 mg/kg IV | G |
| 7 | I + Comp. A | 50 mg/kg IP | 20 mg/kg IV | G |
| 8 | I + Comp. A | 50 mg/kg IP | 40 mg/kg IV | H |
| 9 | I + Comp. A | 50 mg/kg IP | 40 mg/kg IV | J |
| 10 | I + Comp. A | 50 mg/kg IP | 40 mg/kg IV | K |
| 12 | I + berzosertib | 50 mg/kg IP | 60 mg/kg PO | NA |

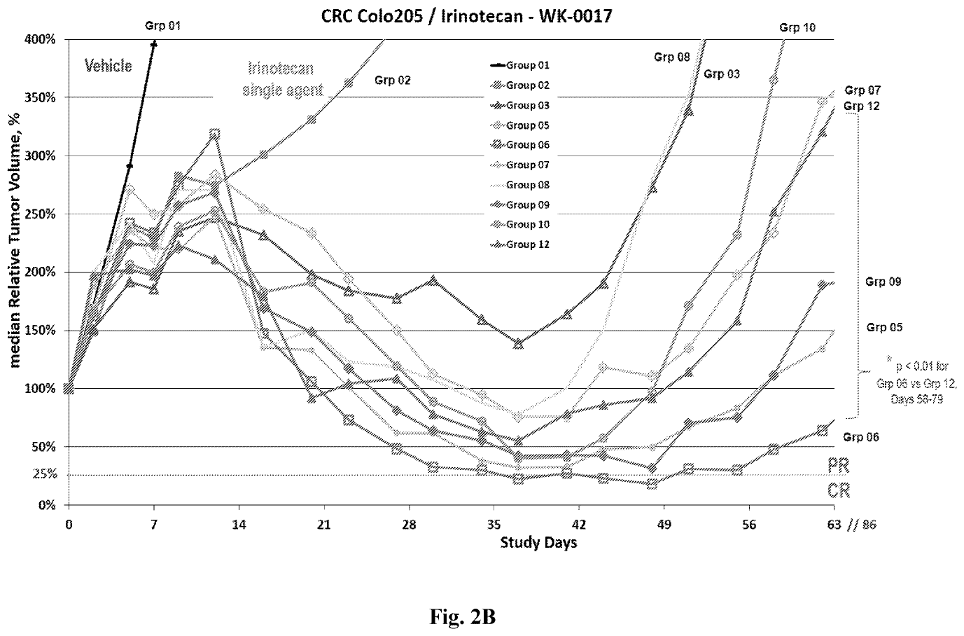

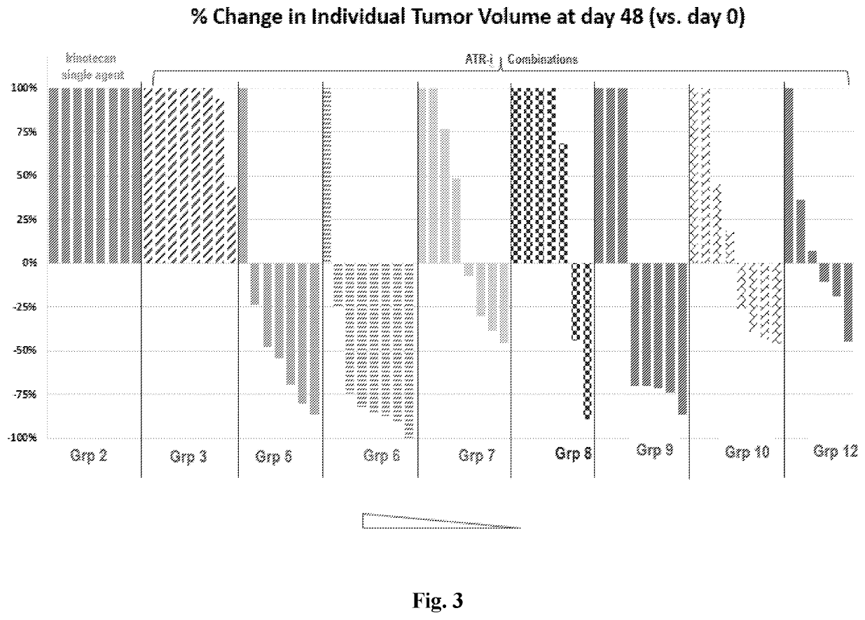

FIG. 2A shows the change in median tumor volume for different study groups as described in Table 2. FIG. 2B is a magnified view of a portion of FIG. 2A. FIG. 3 shows % change in individual tumor volume at day 48 compared to day 0.

Table 3 summarizes the tumor growth inhibition (TGI %) compared to the vehicle (Group 2) and tumor growth dynamic (TGD) compared to irinotecan single agent (Group 2). Table 4 shows the number of mice in each group which achieved complete response (CR) or partial response (PR) in tumor regression.

TABLE 3
Tumor growth inhibition (TGI %)
and tumor growth dynamic (TGD)

| | TGI % (vs. vehicle | | TGD (vs. single agent) | | |
|---|---|---|---|---|---|
| Group | d 20 | d 27 | | % | Index |
| 1 | 0 | 0 | | | |
| 2 | 67 | 74 | | 0 | 1.0 |
| 5 | 85 | 96 | | 94 | 1.9 |
| 6 | 89 | 97 | > | 121 | 2.2 |
| 8 | 86 | 93 | | 45 | 1.4 |
| 9 | 85 | 95 | | 104 | 2.0 |
| 10 | 80 | 92 | | 63 | 1.6 |
| 12 | 91 | 93 | | 85 | 1.8 |

TABLE 4
Number of mice achieving response

| Group | ORR % | CR % | PR# | Total R |
|---|---|---|---|---|
| 1 | 0 | 0 | 0 | 0 |
| 2 | 0 | 0 | 0 | 0 |
| 5 | 62 | 2 | 3 | 6 |
| 6 | 75 | 4 | 2 | 7 |
| 8 | 50 | 1 | 1 | 4 |
| 9 | 62 | 1 | 4 | 5 |
| 10 | 30 | 1 | 2 | 5 |
| 12 | 12 | 0 | 1 | 5 |

ORR = objective response rate;
CR = complete response;
PR = partial response;
R = response

As shown in FIGS. 2A-2B, ATR inhibitors in liposomal formulation in combination with irinotecan enhanced the efficacy when compared to irinotecan as a single agent. Regressions of tumor volume including complete response (CR) and partial response (PR) were observed (Table 4).

Notably, Group 6 displayed sustained complete response tumor regression, surpassing clinical benchmark, berzosertib, with statistical significance. Also, the data demonstrates liposomal ATRi in combination with irinotecan can provide sustained response for over a month after the last dose given at day 33.

### Example 4A. Dosing Schedule Study

Efficacy study similar to those described in Example 3 was repeated where liposomal ATR inhibitors (Compounds A and C) were dosed once a week (instead of every other day). Animal study groups are disclosed in Table 5A (n=8 per group).

TABLE 5A
Study Details

| | | | | ATRi Lipo |
|---|---|---|---|---|
| Group | | | | Formulation |
| No. | Treatment | Dose of I | Dose of ATRi | from Example 1 |
| 1 | Vehicle | none | none | NA |
| 2 | Irinotecan | 50 mg/kg IP | none | NA |
| | (“I”) | | | |
| 3 | I + Comp. C | 50 mg/kg IP | 10 mg/kg IV | Formulation E |
| 4 | I + Comp. C | 50 mg/kg IP | 40 mg/kg IV | Formulation E |
| 5 | I + Comp. C | 50 mg/kg IP | 10 mg/kg IV | Formulation G |
| 6 | I + Comp. C | 50 mg/kg IP | 40 mg/kg IV | Formulation G |
| 7 | I + Comp. A | 50 mg/kg IP | 10 mg/kg IV | Formulation E |
| 8 | I + Comp. A | 50 mg/kg IP | 40 mg/kg IV | Formulation E |

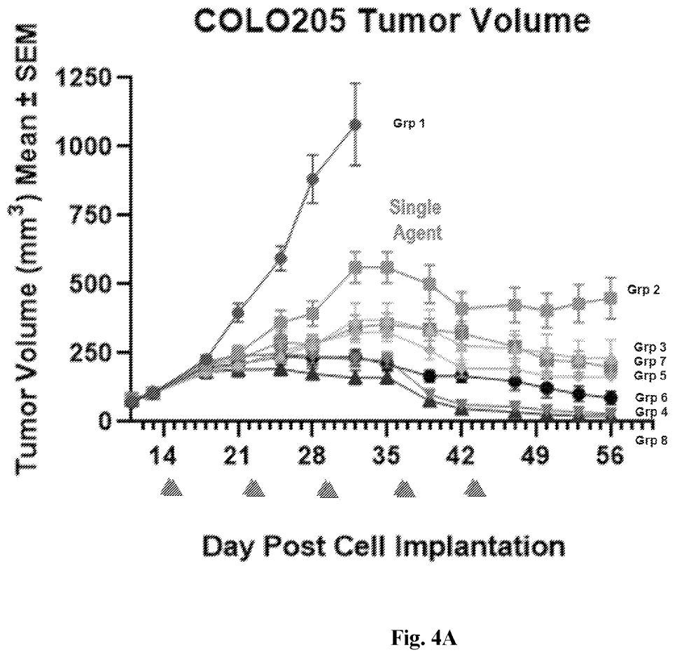

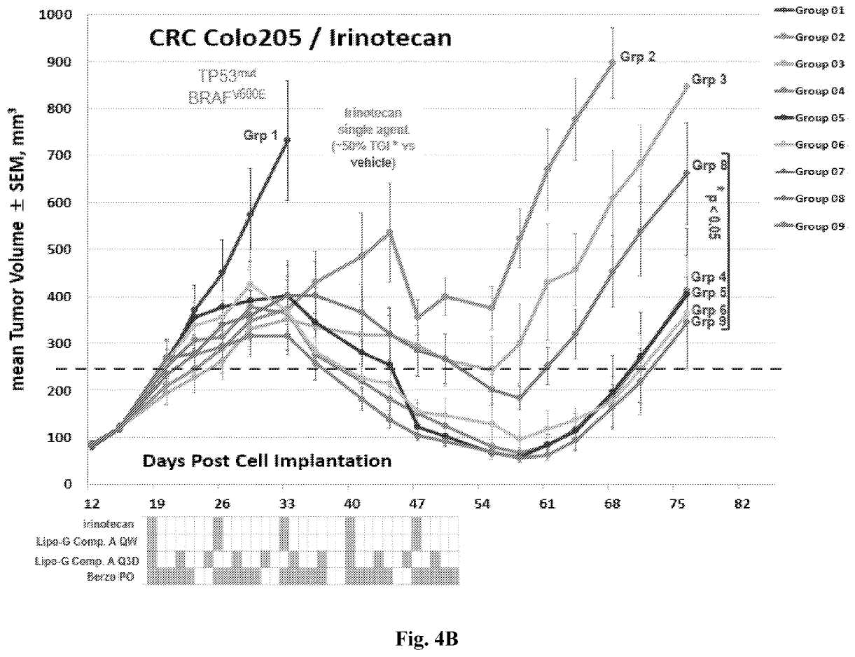

FIG. 4A shows the change in mean tumor volume for different study groups as described in Table 5. Table 6A summarizes the tumor growth inhibition (TGI %) compared to the vehicle (Group 1).

TABLE 6A
tumor growth inhibition (TGI %)

| Group No. | TGI % d 28 (d 14) |
|---|---|
| 1 | 0.0 |
| 2 | 55.4 |
| 3 | 68.5 |
| 4 | 72.9 |
| 5 | 68.0 |
| 6 | 73.5 |
| 7 | 68.2 |
| 8 | 80.2 |

d 28 = Day 28 from tumor cell inoculation into the animal;
d 14 = Day 14 from start of dosing

As shown in FIG. 4A and Table 6A, this study demonstrates that ATR inhibitors in liposomal formulation can be dosed less frequently at once a week to achieve enhanced efficacy compared to irinotecan as a single agent.

### Example 4B. Dosing Schedule Study

Efficacy study similar to those described in Example 3 were also repeated where liposomal ATR inhibitors (Compounds A, C, or berzosertib) were dosed once a week or three times a week. Animal study groups are disclosed in Table 5B (n=8 per group). The liposomal Formulation G from Example 1 is indicated as “Lipo-G” (or “Lipo.G”) and liposomal Formulation E from Example 1 is indicated as “Lipo-E”.

TABLE 5B
Study Details

| Group | | | Dose of ATRi or |
|---|---|---|---|
| No. | Treatment | Dose of I | Berzosertib |
| 1 | Vehicle | none | none |
| 2 | Irinotecan (“I”) | 50 mg/kg IP QW | none |
| 3 | I + Lipo-G Comp. A | 50 mg/kg IP QW | 10 mg/kg IV QW |
| 4 | I + Lipo-G Comp. A | 50 mg/kg IP QW | 40 mg/kg IV QW |
| 5 | I + Lipo-G Comp. A | 50 mg/kg IP QW | 60 mg/kg IV QW |
| 6 | I + Lipo-G Comp. A | 50 mg/kg IP QW | 40 mg/kg IV Q3D |
| 7 | I + Comp. A | 50 mg/kg IP QW | 40 mg/kg IV QW |
| 8 | I + Berzosertib | 50 mg/kg IP QW | 60 mg/kg PO 5 |
| | | | on/2off |
| 9 | I + Lipo-E Comp A | 50 mg/kg IP QW | 40 mg/kg IV QW |

FIG. 4B shows the change in mean tumor volume for different study groups as described in Table 5B. Table 6B summarizes the tumor growth inhibition (TGI %) compared to the vehicle (Group 1).

TABLE 6B
tumor growth inhibition (TGI %)

| Group No. | TGI % D 44(d 12) |
|---|---|
| 1 | — |
| 2 | 0 |
| 3 | 41 |
| 4 | 66 |
| 5 | 53 |
| 6 | 60 |
| 7 | Not tolerated |
| 8 | 40 |
| 9 | 74 |

D 44 = Day 44 from tumor cell inoculation into the animal;
d 12 = Day 12 from start of measurements
*Matched free drug combination at 40 mg/kg was not tolerated.

As shown in FIG. 4B, ATR inhibitors in liposomal formulation in combination with irinotecan enhanced the efficacy when compared to irinotecan as a single agent and demonstrated superiority over clinical benchmark berzosertib near its maximum tolerated dose. Matched free drug combination at 40 mg/kg was not tolerated.

### Example 5. Efficacy of the Combination of Liposomal ATRi and Irinotecan in a Colorectal Cancer Model

An efficacy study similar to those described in Example 3 above, was repeated where liposomal ATR inhibitors (Compounds A) were dosed once a week in a colorectal carcinoma (CRC) model (Colo205). Animal study groups are disclosed in Table 7 (n=8 per group)

TABLE 7
Study details

| Group No. | Treatment | Dose of I | Dose of ATRi |
|---|---|---|---|
| 1 | Vehicle | none | none |
| 2 | Irinotecan (“I”) | 50 mg/kg IP QW | none |
| 3 | Lipo-G Comp. A | none | 10 mg/kg IV QW |
| 4 | Lipo-G Comp. A | none | 20 mg/kg IV QW |
| 5 | Lipo-G Comp. A | none | 40 mg/kg IV QW |
| 6 | I + Lipo-G Comp. A | 50 mg/kg IP QW | 10 mg/kg IV QW |
| 7 | I + Lipo-G Comp. A | 50 mg/kg IP QW | 20 mg/kg IV QW |
| 8 | I + Lipo-G Comp. A | 50 mg/kg IP QW | 40 mg/kg IV QW |
| 9 | I + Comp. A | 50 mg/kg IP QW | 10 mg/kg IV QW |
| 10 | I + Comp. A | 50 mg/kg IP QW | 20 mg/kg IV QW |

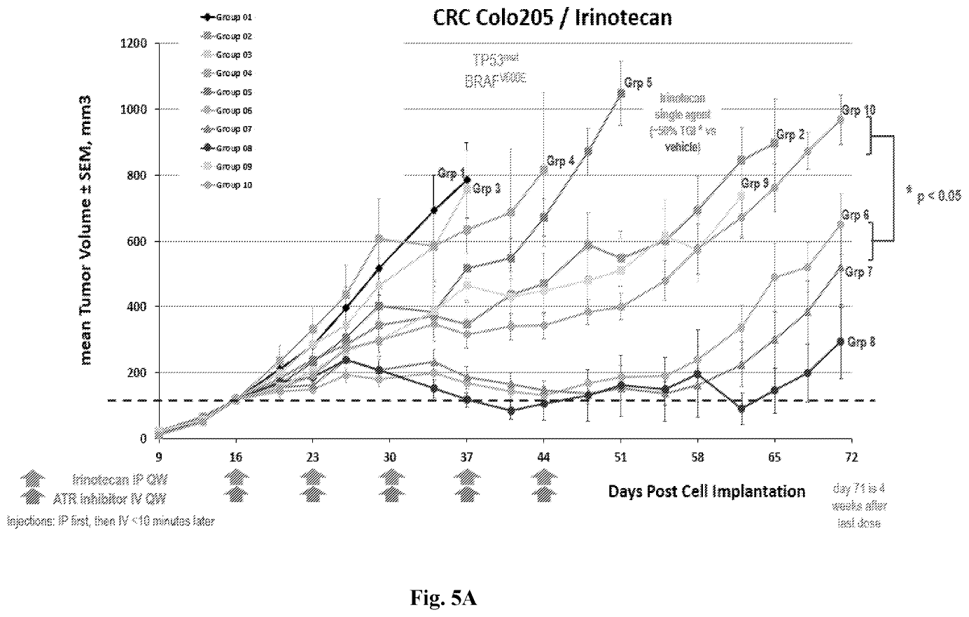

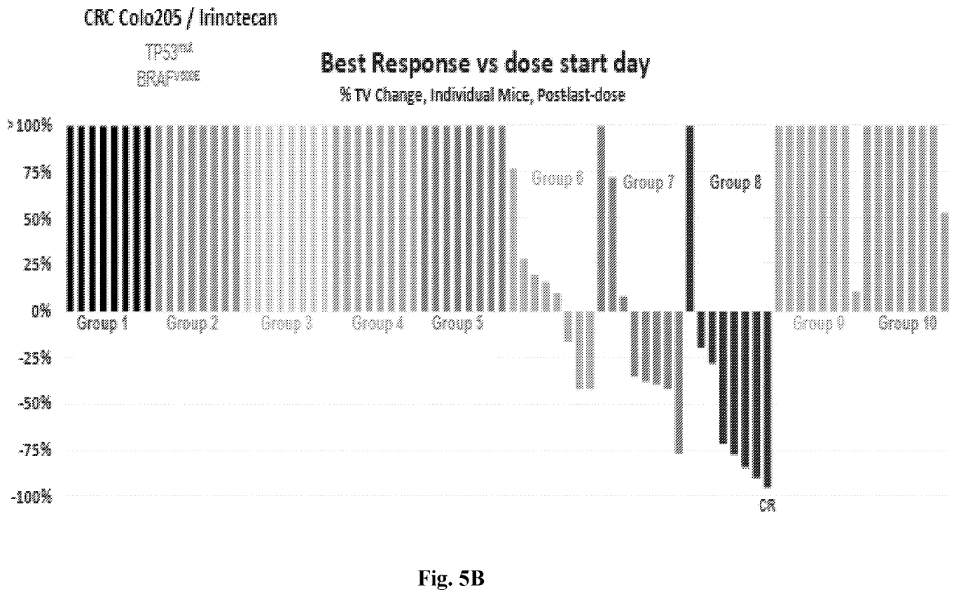

FIG. 5A shows the change in mean tumor volume for different study groups as described in Table 7. FIG. 5B shows % tumor volume change in individual mice, post last dose.

As shown in FIG. 5A all liposome G Compound A formulation in combination with irinotecan showed maximal efficacy. In contrast, no/limited activity was seen with single agents i.e., liposomal Compound A formulation alone and irinotecan alone.

Notably, the combination groups demonstrated durable anti-tumor response after last dose with superiority over clinical benchmark berzosertib near its MTD (maximum tolerated dose).

### Example 6. Efficacy of the Combination of Liposomal ATRi and Irinotecan in a SCLC Model

An efficacy study similar to those described in Example 3 above, was repeated where liposomal ATR inhibitor (Compounds A) and/or irinotecan were dosed once a week in a small-cell lung cancer model (H446) and compared to combinations of irinotecan and berzosertib or irinotecan and ceralasertib. Animal study groups are disclosed in Table 8 (n=8 per group)

TABLE 8
Study details

| Group | | | |
|---|---|---|---|
| No. | Treatment | Dose of I | Dose of ATRi |
| 1 | Vehicle | none | none |
| 2 | Lipo-G Comp. A | none | 50 mg/kg, IV, QW |
| 3 | Lipo-G Comp. A | none | 20 mg/kg, IV, QW |
| 4 | Compound A | none | 20 mg/kg, IV, QW |
| 5 | Irinotecan | 20 mg/kg, IP, QW | None |
| | (“I”) | | |
| 6 | I + Lipo-G | 20 mg/kg, IP, QW | 50 mg/kg, IV, QW |
| | Comp. A | | |
| 7 | I + Lipo-G | 20 mg/kg, IP, QW | 20 mg/kg, IV, QW |
| | Comp. A | | |
| 8 | I + Free | 20 mg/kg, IP, QW | 20 mg/kg, IV, QW |
| | Compound A | | |
| 9 | I + berzosertib | 20 mg/kg, IP, QW | 60 mg/kg, QD, PO |
| 10 | I + ceralasertib | 20 mg/kg, IP, QW | 12.5 mg/kg, |
| | | | BID, PO |

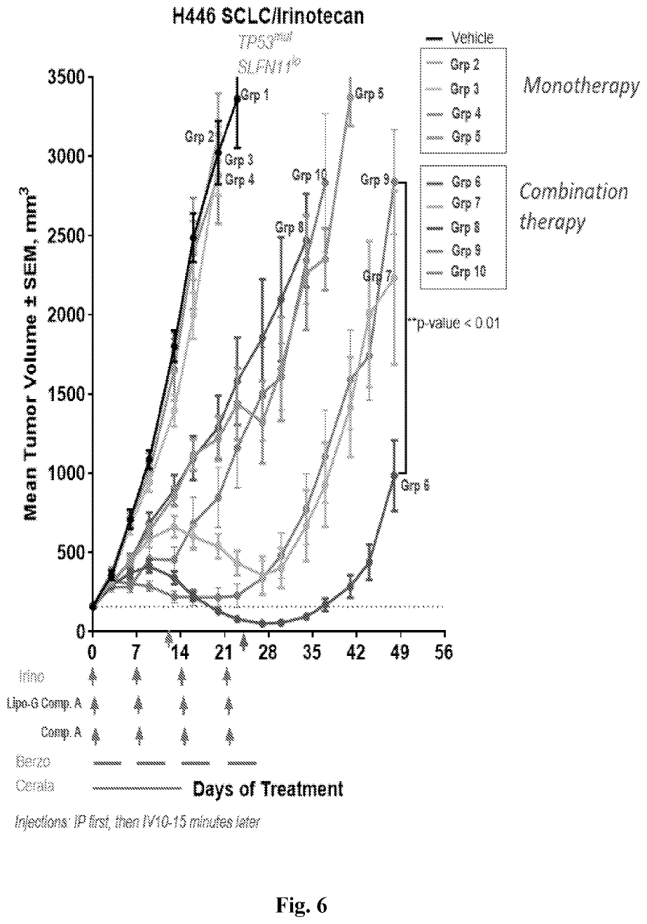

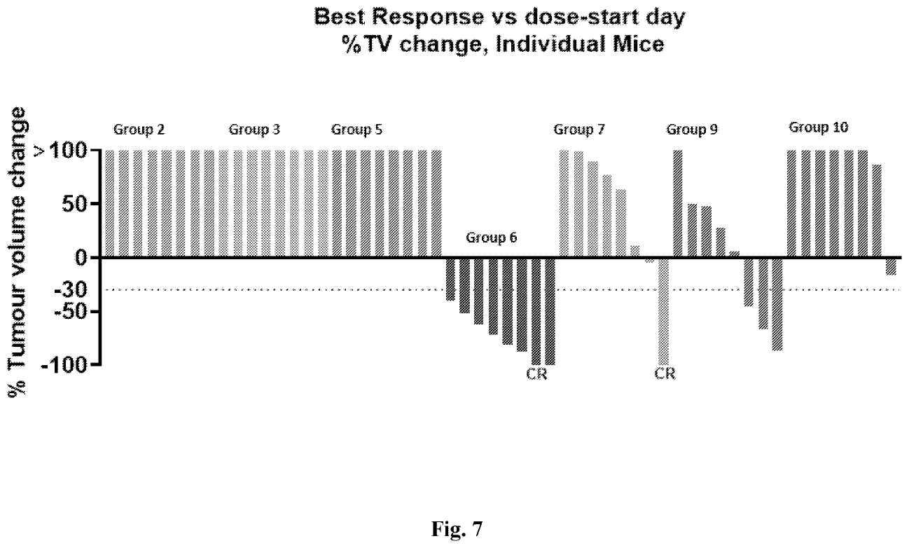

FIG. 6 shows the change in median tumor volume for different study groups as described in Table 8. FIG. 7 shows best response vs dose start day % tumor volume change (% TV) in individual mice.

As shown in FIG. 6 and FIG. 7 liposome G Compound A formulation (group 6) in combination with irinotecan showed superior tumor regression and durable anti-tumor response after the last dose with superiority over both berzosertib and ceralasertib.

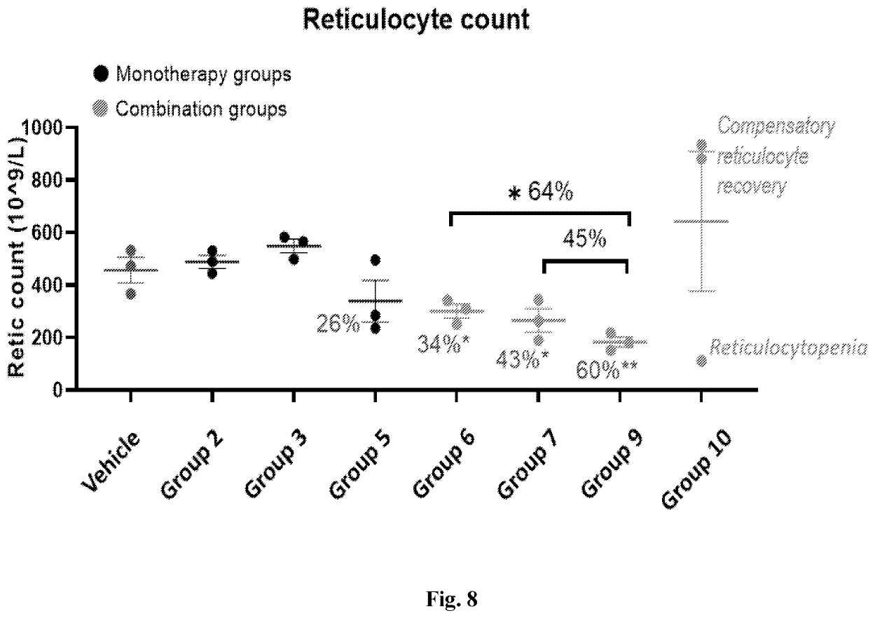

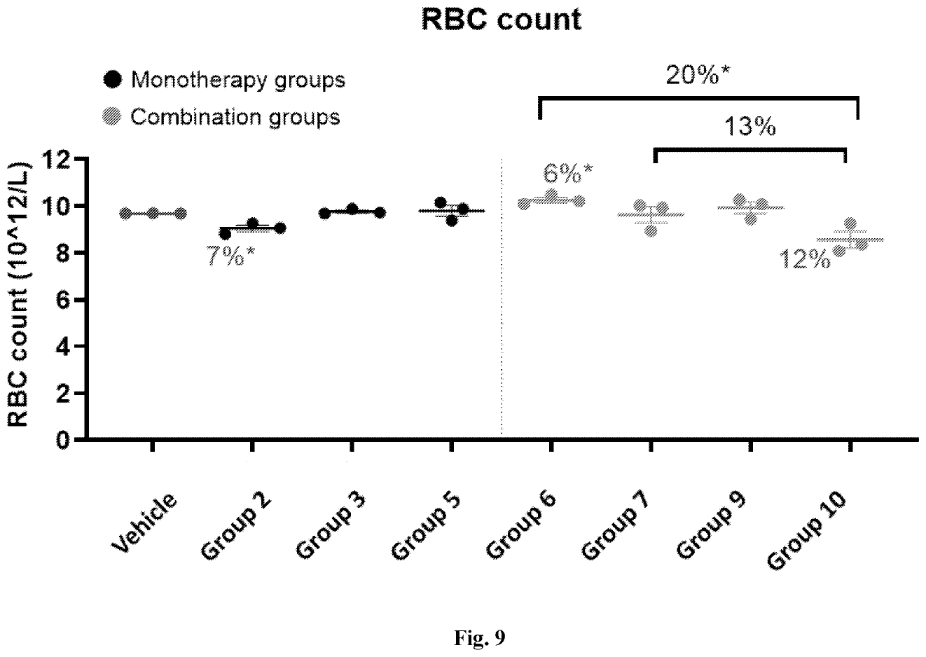

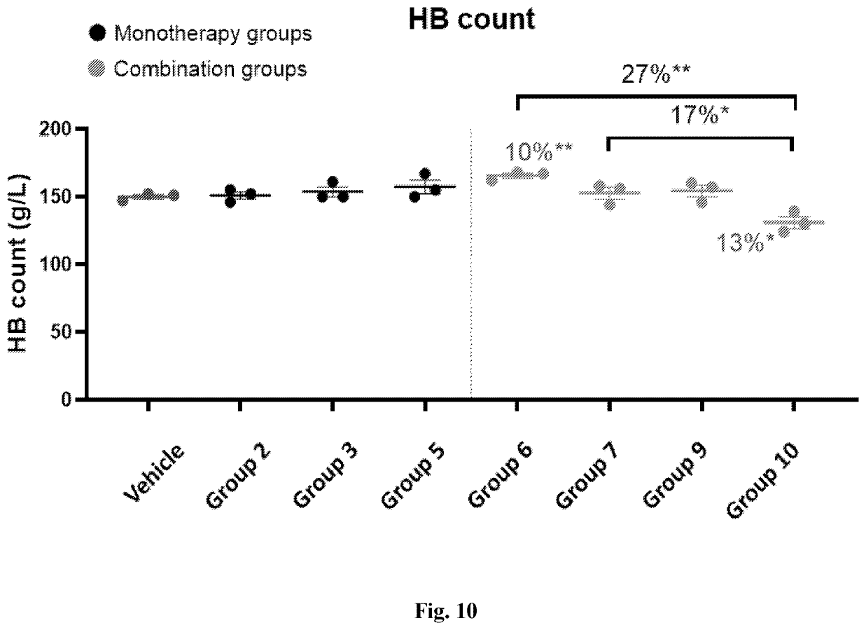

FIGS. 8-10 show that liposomal Compound A exhibited no erythroid toxicity and that notably, the combination of liposomal Compound A and irinotecan exhibited an improved erythroid toxicity profile compared to the clinical benchmark (berzosertib or ceralasertib) in H446 xenograft.

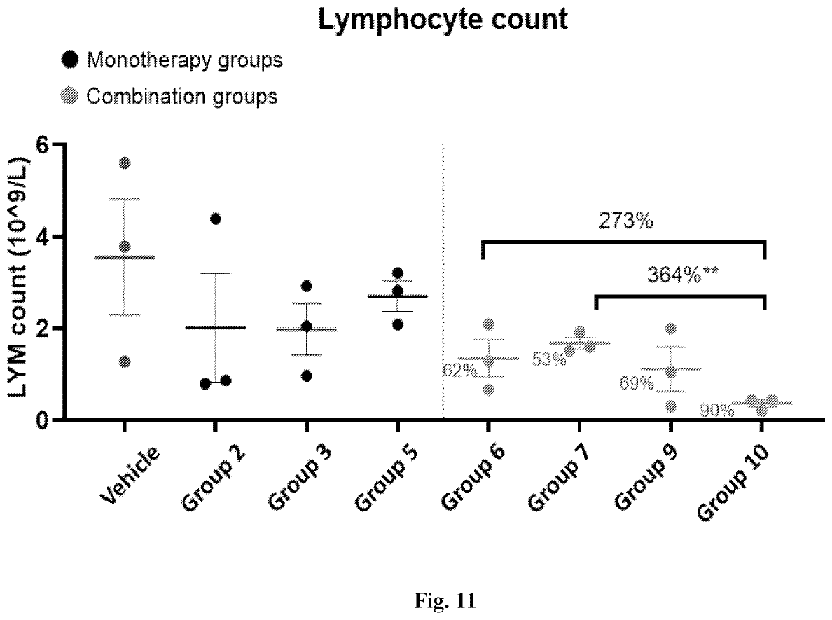

FIG. 11 shows that that liposomal Compound A exhibited no significant lymphocyte toxicity and that the combination of liposomal Compound A and irinotecan exhibited an improvement in the lymphocyte toxicity profile compared to clinical benchmark (berzosertib or ceralasertib) in H446 xenograft.

A study was also conducted where liposomal ATR inhibitor (Compound A) was administered at 50 mg/kg IV once per week in combination with irinotecan administered at 20 mg/kg IP once per week and compared to different doses of irinotecan in a small-cell lung cancer model (H446). Animal study groups are disclosed in Table 9 (n=8).

TABLE 9
Study details

| Group No. | Treatment | Dose of I | | Dose of ATRi |
|---|---|---|---|---|
| 1 | Vehicle | none | | none |
| 2 | Irinotecan (“I”) | 5 | mg/kg | None |
| 3 | Irinotecan | 10 | mg/kg | None |
| 4 | Irinotecan | 20 | mg/kg | None |
| 5 | Irinotecan | 40 | mg/kg | None |
| 6 | Irinotecan + Lipo-G Comp. A | 20 | mg/kg | 50 mg/kg |

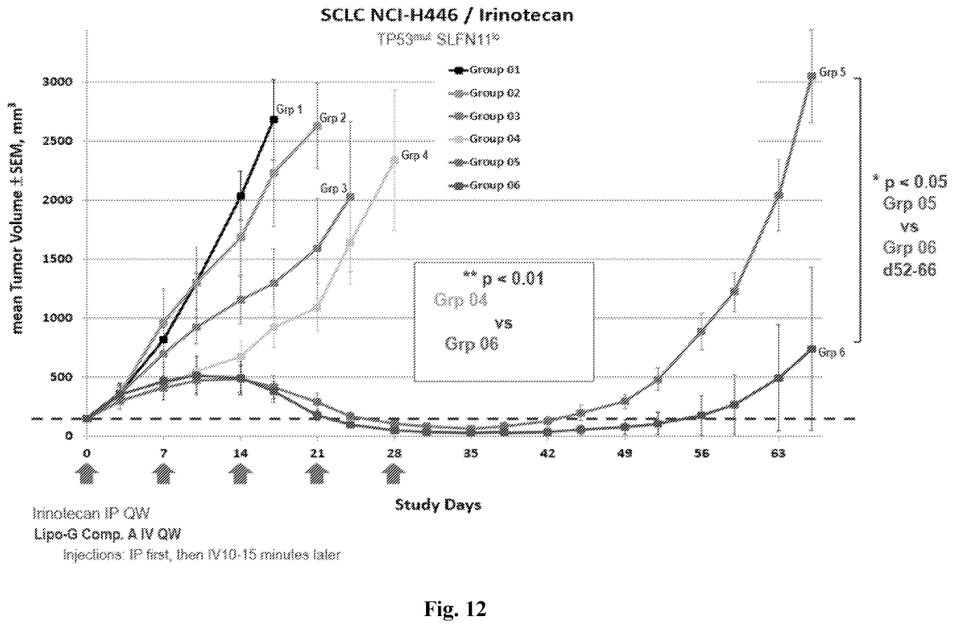

FIG. 12 shows mean tumor volume for different study groups as described in Table 9.

As shown in FIG. 12 the combination therapy demonstrated durable anti-tumor response and was significantly superior to irinotecan monotherapy.

### Example 7. Efficacy of the Combination of Liposomal ATRi and Irinotecan in a NSCLC Model

An efficacy study similar to those described in Example 3 above, was repeated where liposomal ATR inhibitor (Compounds A) and/or irinotecan were dosed at 50 mg/kg once a week in a non-small-cell lung cancer model (CTG-0828 PDX) for 5 weeks (QWx5) and compared to combinations of liposomal ATR inhibitor (Compounds A) and irinotecan. Animal study groups are disclosed in Table 10 (n=8 per group).

TABLE 10
Study details

| Group No. | Treatment | Dose of I | Dose of ATRi |
|---|---|---|---|
| 1 | Vehicle | none | none |
| 2 | Irinotecan (“I”) | 50 mg/kg | none |
| 3 | Lipo-G Comp. A | none | 50 mg/kg |
| 4 | Irinotecan + Lipo-G Comp. A | 50 mg/kg | 50 mg/kg |

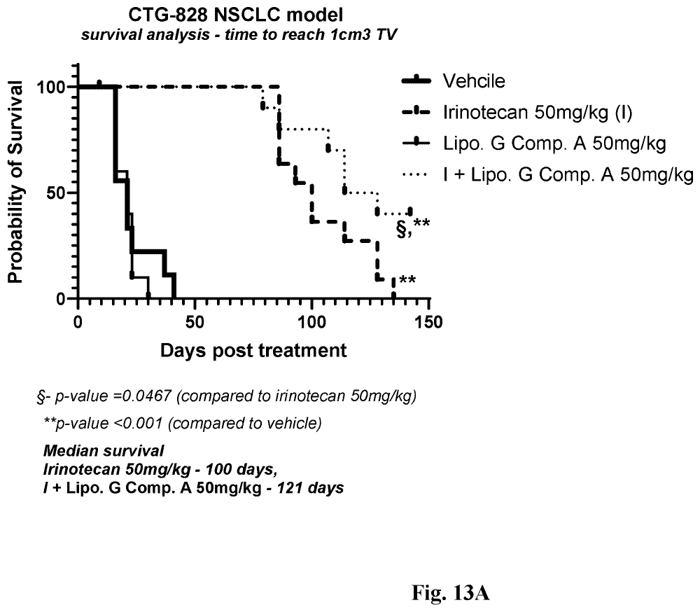

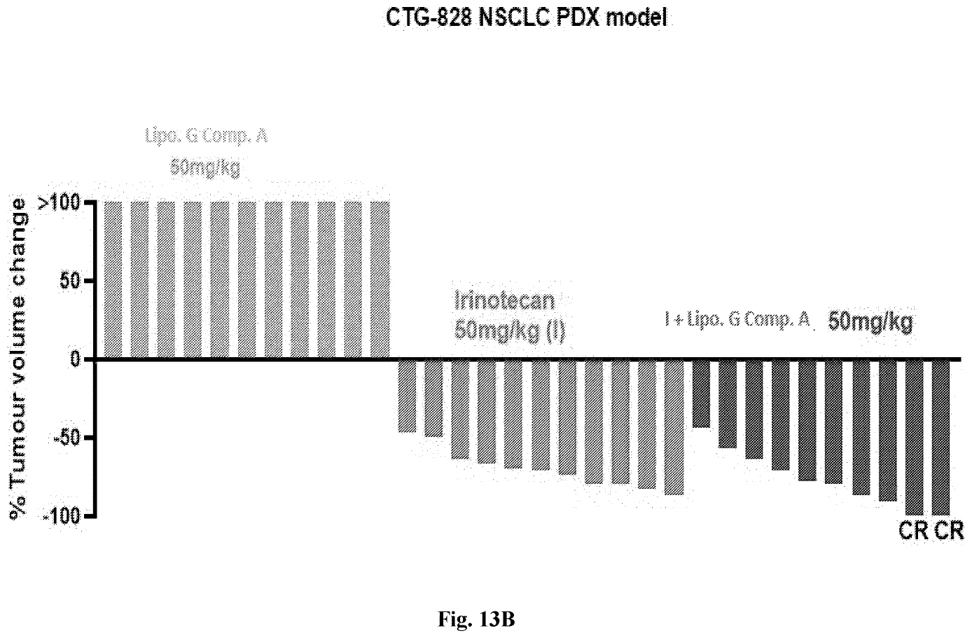

FIG. 13A shows the probability of survival of different study groups as shown in Table 10. FIG. 13B shows the percent change in tumor volume for study groups 2-4 as shown in Table 10.

As shown in the FIGS. 13A and 13B as well as Table 11 below, the liposomal formulation of Compound A versus irinotecan monotherapy showed i) a Tumor Growth Delay Index (TGDI) of 5.3 vs. 4.4, respectively; ii) a statistically significant improvement in survival (reading out as median time to reach and exceed a tumor burden of 1 cm), 121 days vs. 100 days, respectively; and iii) 20% vs 0% CR tumor regression rate, respectively.

TABLE 11
Treatment results

| | Max % Tumor | | | |
|---|---|---|---|---|
| | Grown | TGDI | TGDI | Regression |
| | Inhibition | (vs. | (vs. | * |
| Treatment | (TGI) | vehicle) | irinotecan) | (% max) |
| Irinotecan 50 | 91 | 4.4 | — | 60 |
| mg/kg (I) | | | | |
| Lipo-G Comp. A | 0 | 0.9 | 0.2 | 0 |
| 50 mg/kg | | | | |
| I + Lipo-G. Comp. | 92 | 5.3 | 1.2 | 65 |
| A 50 mg/kg | | | | |

### Example 8. Efficacy of the Combination of Liposomal ATRi and Irinotecan in an Ovarian Cancer Model

Subcutaneous tumor models were initiated on 6-8 week old, female NOD-scid IL2Rgammanull (NSG) mice. A cell suspension containing 1.5 million OVCAR8 cells in 1:1 PBS: Matrigel was subcutaneously injected into the hind flank of NSG mice. Tumors were measured twice weekly by digital caliper and reported as volume, calculated as V=π×1×w∧2 wherein 1 and w denote the longest and shortest tumor diameter respectively. Engrafted mice were then randomized into cohorts for treatment at an average tumor size of 200 mm³.

Orthotopic tumor xenografts were conducted on post-pubescent, 10-13 week old, female NSG mice. 2 million bioluminescent OVCAR8 cells suspended in 500 μL of PBS were inoculated into the intraperitoneal cavity of NSG mice. Diseased mice were then randomized into cohorts and treated at 2-weeks post-inoculation. Tumor burden was assessed by once-weekly in vivo bioluminescent imaging (BLI) and reported as total flux (p/s).

Patient derived xenograft models were established in collaboration with the Princess Margaret Living Biobank. Patient tumor samples were collected, sectioned, and transplanted into the flank of female BALB/c mice. Tumors were then passaged through host BALB/c mice between a minimum of 3-5 passages prior to collection and SC inoculation onto 6-8 week old, female NSG mice. Tumor measurements were taken by digital caliper and reported as volume as described above. Tumors were then grown to an average of 200 mm³ before randomization into evaluated cohorts.

Table 12 shows the treatment schedule for the efficacy studies (n=5; dosed three times per week at every-other-day with a two-day weekend holiday). Animal study groups are disclosed in Table 11 (n=5 per group).

TABLE 12
Study details

| Group No. | Treatment | Dose of I | Dose of ATRi |
|---|---|---|---|
| 1 | Vehicle | none | none |
| 2 | Lipo-G Comp. A | none | 80 mg/kg |
| 3 | Irinotecan | 50 mg/kg | none |
| 4 | Lipo-G Comp. A + | 50 mg/kg | 50 mg/kg |
| | Irinotecan | | |

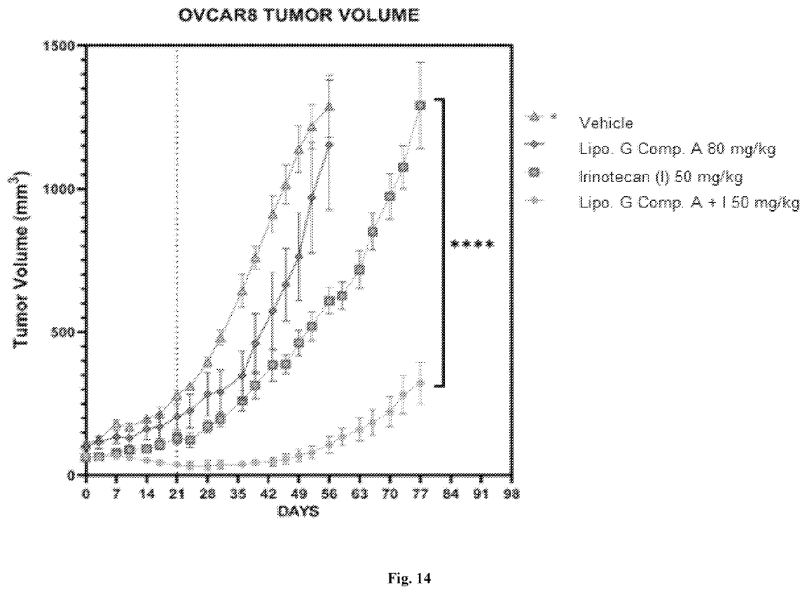

FIG. 14 shows shows the change in OVCAR8 tumor volume (mm³) after treatment of each study group on Table 12.

### Example 9. Drug Tolerance Studies

Maximum tolerated dose studies are conducted on healthy, 6-8 week-old athymic nude mice (Jackson Laboratories). For ATRi tolerability, ATR inhibitor of formula (I) both in free and liposomal form is IV administered to nude mice at a dosing schedule of every other day followed by a two-day holiday (i.e., three days per week) for 4-weeks. Study mice are then sacrificed at 24-hours post final injection with endpoint blood collection by cardiac puncture. Complete blood count (CBC) is then conducted on a Vetscan HM5 hematology analyzer (Zoetis, New Jersey, USA).

Systemic toxicity in drug administered mice is expressed as weight change and survival over a 2-4 weeks study duration, respectively. CBC analysis after free ATRi or liposomal ATRi administration, over a 2-4 weeks study duration is reported.

### Example 10. Hematology Tolerability Study

In order to study the effects of hematology tolerability under different dosing regimens, 8-12 week old, female C57BL/6 mice are used. Body weight is measured qd×5, then biweekly to the end of the study. Animals are weighed and monitored for survival and in-life observations are collected at the time of weight measurement. Mice are organized into appropriate groups consisting of control (empty vehicle) and treatment groups including free irinotecan, irinotecan plus berzosertib, irinotecan plus elimusertib and a combination of irinotecan and liposomal Compound A. Mice are given orally, or injected intravenously, with the required volume of sample to administer the prescribed dose to the animals based on individual mouse weights on dosing days. Mice are monitored individually to endpoint which is 18 days. Full volume blood is collected by terminal cardiac puncture under isoflurane anesthesia and analyzed for reticulocyte count/number, red blood cell count/number, hemoglobin, hematocrit, platelet count/number, white blood cell count/number, neutrophil count/number, monocyte count/number and lymphocyte count/number.

The publications discussed herein are provided solely for their disclosure prior to the filing date of the present application. Nothing herein is to be construed as an admission that the present invention is not entitled to antedate such publication by virtue of prior invention. While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### EMBODIMENTS

Embodiment 1. A liposomal composition comprising:
(i) a compound of formula (I):

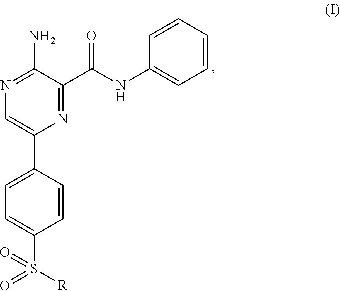

or a pharmaceutically acceptable salt thereof, wherein
R is a moiety comprising an amino group with a pKₐ of greater than 7.0; and
(ii) a liposome comprising a phospholipid, a cholesterol, and a PEG-lipid conjugate; wherein the composition is stable for at least two weeks.

Embodiment 2. The composition of embodiment 1, wherein:
R is —C₁-C₄ alkylene-NR^{a}R^{b}, —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, —(C₁-C₄ alkylene)-(heterocyclyl)-NR^{a}R^{b}, or —NR^{a}R^{b}—(C₁-C₄ alkyl), wherein the heterocyclyl contains at least one nitrogen as a ring atom; and
R^{a} and R^{b} are each independently H or —C₁-C₄ alkyl.

Embodiment 3. The composition of embodiment 1 or 2, wherein R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

Embodiment 4. The composition of any one of embodiments 1-3, wherein R is:
a)

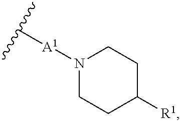

wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkylene)-NR^{a}R^{b}, wherein R^{a} and R^{b} are each independently H or —C₁-C₄ alkyl;
c) —(C₁-C₈ alkyl)-NR^{a}R^{b}; wherein R^{a} and R^{b} are each independently H or C₁-C₄ alkyl; or
d)

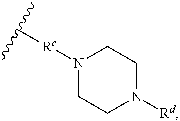

wherein R^{c} is —C₁-C₄ alkylene and R^{d} is —C₁-C₄ alkyl.

Embodiment 5. The composition of any one of embodiments 1-4, wherein the compound of formula (I) is

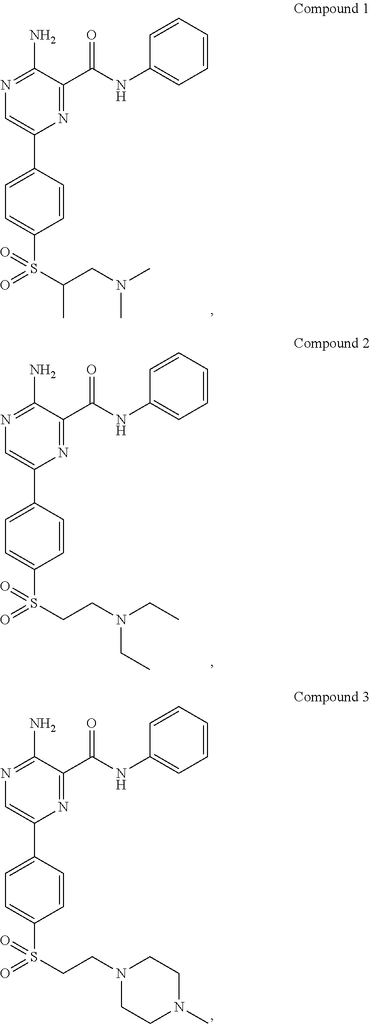 [SMILES: CC(CN(C)C)S(=O)(=O)c1ccc(-c2cnc(N)c(C(=O)Nc3ccccc3)n2)cc1 ; CCN(CC)CCS(=O)(=O)c1ccc(-c2cnc(N)c(C(=O)Nc3ccccc3)n2)cc1 ; CN1CCN(CCS(=O)(=O)c2ccc(-c3cnc(N)c(C(=O)Nc4ccccc4)n3)cc2)CC1]

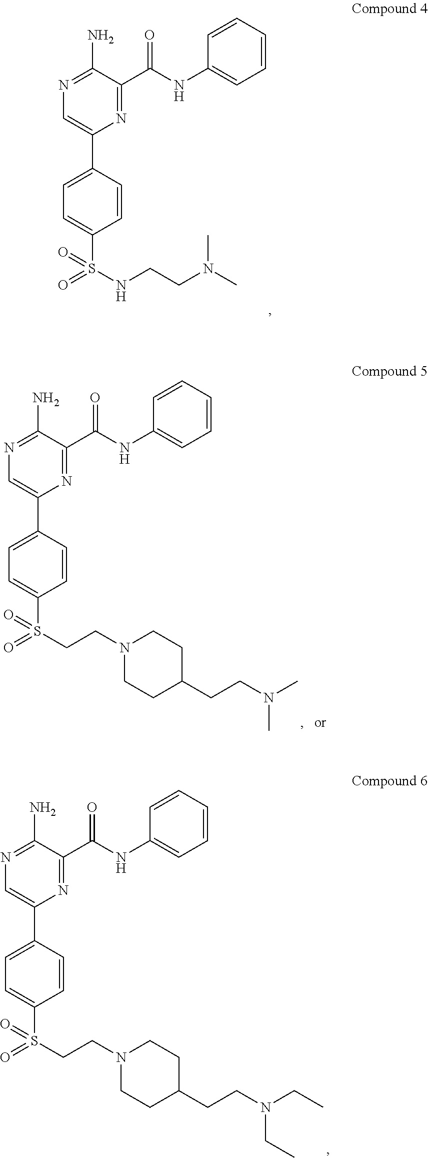 [SMILES: CN(C)CCNS(=O)(=O)c1ccc(-c2cnc(N)c(C(=O)Nc3ccccc3)n2)cc1 ; CN(C)CCC1CCN(CCS(=O)(=O)c2ccc(-c3cnc(N)c(C(=O)Nc4ccccc4)n3)cc2)CC1 ; CCN(CC)CCC1CCN(CCS(=O)(=O)c2ccc(-c3cnc(N)c(C(=O)Nc4ccccc4)n3)cc2)CC1]

or a pharmaceutically acceptable salt thereof.

Embodiment 6. The composition of any one of embodiments 1-5, wherein the composition further comprises another pharmaceutically active agent.

Embodiment 7. The composition of embodiment 7, wherein the other pharmaceutically active agent is irinotecan.

Embodiment 8. The composition of any one of embodiments 1-7, wherein the phospholipid is 1,2-distearoyl-sn-glycero-3-phosphocholine (DSPC).

Embodiment 9. The composition of any one of embodiments 1-8, wherein the PEG-lipid conjugate is PEG (2000)-distearoylglycerol (PEG-DSG).

Embodiment 10. The composition of any one of embodiments 1-9 wherein the liposome comprises about 50 mol % to about 70 mol % of DSPC.

Embodiment 11. The composition of embodiment 20, wherein the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

Embodiment 12. The composition of any one of embodiments 1-11, wherein the liposome comprises about 30 mol % to about 50 mol % of cholesterol.

Embodiment 13. The composition of any one of embodiments 1-12, wherein the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

Embodiment 14. The composition of any one of embodiments 1-13, wherein the liposome comprises about 0.1 mol % to about 5 mol % of PEG-DSG.

Embodiment 15. The composition of embodiment 14, wherein the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

Embodiment 16. The composition of any one of embodiments 1-15, wherein the liposome comprises DSPC, cholesterol, and PEG-DSG in molar ratio of about 58:40:2 or about 59.75:40:0.25.

Embodiment 17. The composition of any one of embodiments 1-16, wherein the composition is stable for at least 6 months.

Embodiment 18. A method for treating cancer, comprising administering to a subject in need thereof an effective amount of the composition of any one of embodiments 1-17.

Embodiment 19. The method of embodiment 18, wherein the cancer is breast cancer, ovarian cancer, pancreatic cancer, prostate cancer, colorectal cancer, lung cancer, brain cancer, melanoma, or osteosarcoma.

Embodiment 20. The method of embodiment 19, wherein the breast cancer is triple negative breast cancer.

Embodiment 21. The method of embodiment 19, wherein the lung cancer is small cell lung cancer or non-small cell lung cancer.

Embodiment 22. The method of any one of embodiments 17-21, wherein the method does not cause acute neurological toxicity.

Embodiment 23. The method of any one of embodiments 17-22, wherein the method does not cause long-term hematological toxicity.

Embodiment 24. The method of any one of embodiments 17-23, wherein the method provides sustained systemic exposure of the compound of formula (I) or a pharmaceutically acceptable salt thereof for at least 2 days, at least 3 days, or at least 4 days after a single dose administration.

Embodiment 25. The method of any one of embodiments 1-23, wherein the composition is administered to the subject once every 2 days, once every 3 days, once every 4 days, once every 5 days, once every 6 days, or once every 7 days.

Embodiment 26. The method of any one of embodiments 1-23, wherein the composition is administered to the subject once a week or once every 2 weeks.

What is claimed is:

1. A method of treating cancer in a subject in need thereof, comprising administering a liposomal composition comprising:
(i) a compound of formula (I): 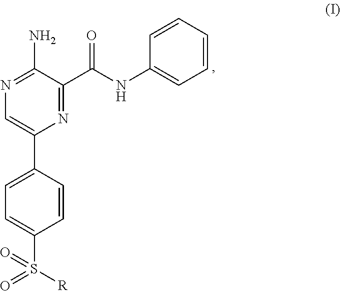
or a pharmaceutically acceptable salt thereof, wherein
R is a moiety comprising an amino group with a pKₐ of greater than 7.0; and
(ii) a liposome comprising 1,2-distearoyl-sn-glycero-3-phosphocholine (DSPC) and a cholesterol.

2. The method of claim 1, wherein:
R is —C₁-C₄ alkylene-NR^{a}R^{b}, —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, —(C₁-C₄ alkylene)-(heterocyclyl)-NR^{a}R^{b}, or —NR^{a}R^{b}—(C₁-C₄ alkyl), wherein the heterocyclyl contains at least one nitrogen as a ring atom; and
R^{a} and R^{b} are each independently H or —C₁-C₄ alkyl.

3. The method of claim 1 or 2, wherein R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

4. The method of any one of claims 1-3, wherein R is:
a) 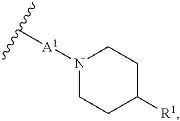
wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkylene)-NR^{a}R^{b}, wherein R^{a} and R^{b} are each independently H or —C₁-C₄ alkyl;
c) —(C₁-C₈ alkyl)-NR^{a}R^{b}, wherein R^{a} and R^{b} are each independently H or C₁-C₄ alkyl; or
d) 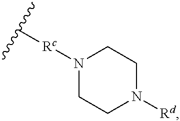
wherein R^{c} is —C₁-C₄ alkylene and R^{d} is —C₁-C₄ alkyl.

5. The method of any one of claims 1-4, wherein the compound of formula (I) is 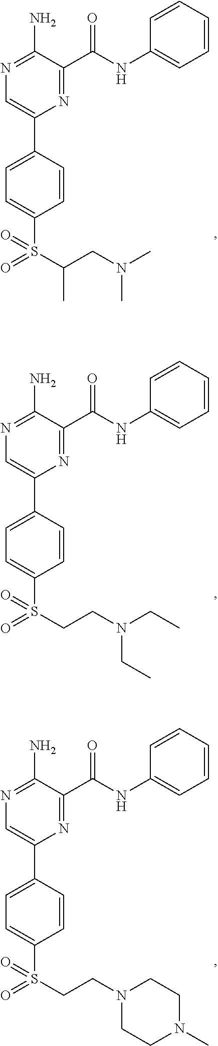 [SMILES: CC(CN(C)C)S(=O)(=O)c1ccc(-c2cnc(N)c(C(=O)Nc3ccccc3)n2)cc1 ; CCN(CC)CCS(=O)(=O)c1ccc(-c2cnc(N)c(C(=O)Nc3ccccc3)n2)cc1 ; CN1CCN(CCS(=O)(=O)c2ccc(-c3cnc(N)c(C(=O)Nc4ccccc4)n3)cc2)CC1] 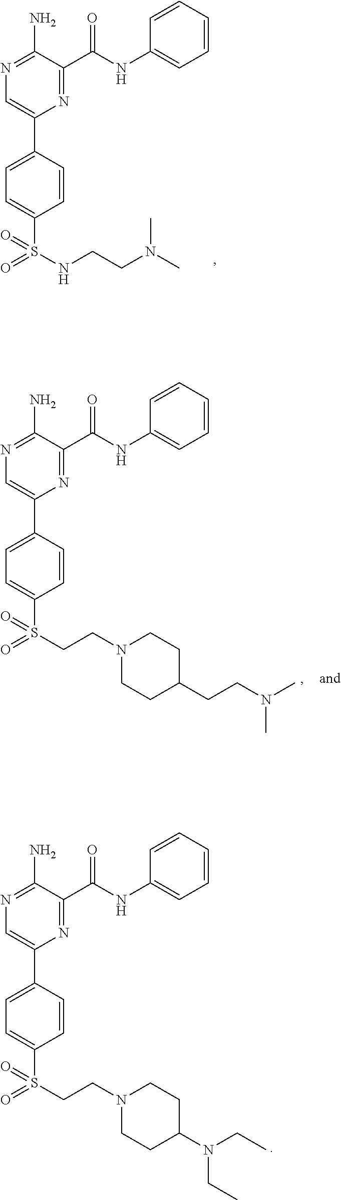 [SMILES: CN(C)CCNS(=O)(=O)c1ccc(-c2cnc(N)c(C(=O)Nc3ccccc3)n2)cc1 ; CN(C)CCC1CCN(CCS(=O)(=O)c2ccc(-c3cnc(N)c(C(=O)Nc4ccccc4)n3)cc2)CC1 ; CCN(CC)C1CCN(CCS(=O)(=O)c2ccc(-c3cnc(N)c(C(=O)Nc4ccccc4)n3)cc2)CC1]
or a pharmaceutically acceptable salt thereof.

6. The method of any one of claims 1-5, wherein the subject is a mammal.

7. The method of any one of claims 1-6, wherein the liposome comprises a PEG-lipid conjugate.

8. The method of claim 7, wherein the PEG-lipid conjugate is PEG (2000)-distearoylglycerol (PEG-DSG).

9. The method of any one of claims 1-8 wherein the liposome comprises about 50 mol % to about 70 mol % of DSPC.

10. The method of claim 9, wherein the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

11. The method of any one of claims 1-10, wherein the liposome comprises about 30 mol % to about 50 mol % of cholesterol.

12. The method of any one of claims 1-11, wherein the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

13. The method of any one of claims 8-12, wherein the liposome comprises about 0.1 mol % to about 5 mol % of PEG-DSG.

14. The method of claim 13, wherein the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

15. The method of any one of claims 8-14, wherein the liposome comprises about 0.1 mol % to about 2.5 mol % PEG-DSG.

16. The method of any one of claims 8-15, wherein the liposome comprises about 0.1 mol % to about 2 mol % PEG-DSG.

17. The method of any one of claims 8-16, wherein the liposome comprises about 0.1 mol % to about 0.5 mol % PEG-DSG.

18. The method of any one of claims 8-14, wherein the liposome comprises DSPC, cholesterol, and PEG-DSG in molar ratio of about 58:40:2 or about 59.75:40:0.25.

19. The method of any one of claims 1-18, wherein the composition is stable for at least two weeks

20. The method of any one of claims 1-19, wherein the composition is stable for at least 6 months.

21. The method of any one of claims 1-20, wherein the cancer is a solid tumor.

22. The method of any one of claims 1-21, wherein the cancer is ovarian cancer, pancreatic cancer, prostate cancer, brain cancer, melanoma, gastric cancer, or endometrium cancer.

23. The method of any one of claims 1-21, wherein the cancer is colorectal cancer, cervical cancer, lung cancer, triple negative breast cancer, or osteosarcoma.

24. The method of claim 22, wherein the cancer is ovarian cancer.

25. The method of any one of claims 1-24, wherein the method does not cause acute neurological toxicity.

26. The method of any one of claims 1-25, wherein the method does not cause long-term hematological toxicity.

27. The method of any one of claims 1-26, wherein the method provides sustained systemic exposure of the compound of formula (I) or a pharmaceutically acceptable salt thereof for at least 2 days, at least 3 days, or at least 4 days after a single dose administration.

28. The method of any one of claims 1-27, wherein the composition is administered to the subject once every 2 days, once every 3 days, once every 4 days, once every 5 days, once every 6 days, or once every 7 days.

29. The method of any one of claims 1-27, wherein the composition is administered to the subject once a week or once every 2 weeks.

30. The method of any one of claims 1-29, wherein the method further comprises administering another pharmaceutically active agent.

31. The method of claim 30, wherein the other pharmaceutically active agent is an anti-cancer agent.

32. The method of claim 30 or 31, wherein the other pharmaceutically active agent is irinotecan or topotecan.

33. The method of claim 30 or 31, wherein the other pharmaceutically active agent is a platinum agent.

34. The method of claim 33, wherein the platinum agent is cisplatin or carboplatin.

35. The method of claim 30 or 31, wherein the other pharmaceutically active agent is gemcitabine.

36. The method of any one of claims 30-35, wherein the other pharmaceutically active agent is administered in a separate pharmaceutical composition from the liposomal composition comprising the compound of formula (I) or a pharmaceutically acceptable salt thereof.

37. The method of claim 36, wherein the separate pharmaceutical composition is not a liposomal formulation.

38. The method of any one of claims 30-37, wherein the liposomal composition comprising the compound of formula (I) or a pharmaceutically acceptable salt thereof and the other pharmaceutically active agent is administered to the subject separately.

39. A liposomal composition comprising:
(i) a compound of formula (I): 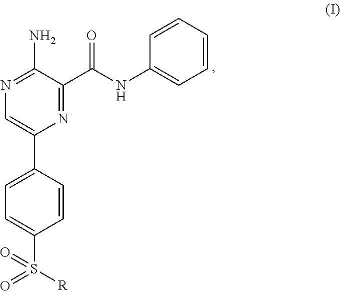
or a pharmaceutically acceptable salt thereof, wherein
R is a moiety comprising an amino group with a pKₐ of greater than 7.0; and
(ii) a liposome comprising a phospholipid, a cholesterol, and a PEG-lipid conjugate;
wherein the composition is stable for at least two weeks.

40. A liposomal composition comprising:
(i) a compound of formula (I): 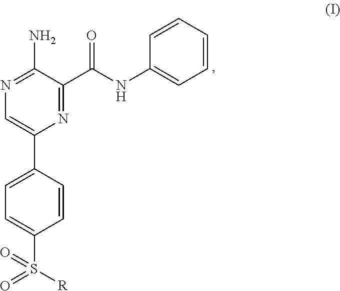
or a pharmaceutically acceptable salt thereof, wherein
R is a moiety comprising an amino group with a pKₐ of greater than 7.0; and
(ii) a liposome comprising a 1,2-distearoyl-sn-glycero-3-phosphocholine (DSPC), and a cholesterol.

41. The liposome composition of claim 41, wherein the liposome comprises a PEG-lipid conjugate.

42. A liposomal composition comprising:
(i) a compound of formula (I): 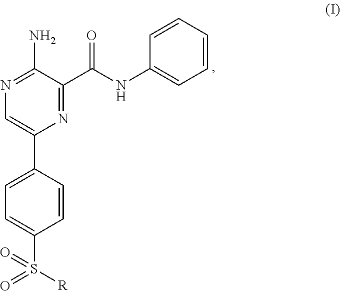
or a pharmaceutically acceptable salt thereof, wherein
R is a moiety comprising an amino group with a pKₐ of greater than 7.0; and
(ii) a liposome comprising a phospholipid, a cholesterol, and a PEG-DSG;
wherein the liposome comprises about 0.1 mol % to about 2 mol % PEG-DSG.

43. The composition of any one of claims 39-42, wherein:
R is —C₁-C₄ alkylene-NR^{a}R^{b}, —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, —(C₁-C₄ alkylene)-(heterocyclyl)-NR^{a}R^{b}, or —NR^{a}R^{b}—(C₁-C₄ alkyl), wherein the heterocyclyl contains at least one nitrogen as a ring atom; and
R^{a} and R^{b} are each independently H or —C₁-C₄ alkyl.

44. The composition of any one of claims 39-43, wherein R is —(C₁-C₄ alkylene)-(heterocyclyl)-(C₁-C₄ alkyl), —(C₁-C₄ alkylene)-(heterocyclyl)-(C₁-C₄ alkylene)-NR^{a}R^{b}, or —(C₁-C₄ alkylene)-(heterocyclyl)-NR^{a}R^{b}, wherein the heterocyclyl is a piperidine or a piperazine.

45. The composition of any one of claims 39-44, wherein R is:
a) 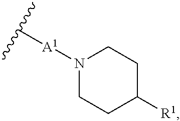
wherein A¹ is absent or C₁-C₄ alkylene, and R¹ is —(C₁-C₄ alkyl)-NR^{a}R^{b}, wherein R^{a} and R^{b} are each independently H or —C₁-C₄ alkyl;
b) —N(R^{a})(C₁-C₄ alkylene)-NR^{a}R^{b}, wherein R^{a} and R^{b} are each independently H or —C₁-C₄ alkyl;
c) —(C₁-C₈ alkyl)-NR^{a}R^{b}, wherein R^{a} and R^{b} are each independently H or C₁-C₄ alkyl; or
d) 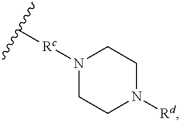
wherein R^{c} is —C₁-C₄ alkylene and R^{d} is —C₁-C₄ alkyl.

46. The composition of any one of claims 39-45, wherein the compound of formula (I) is 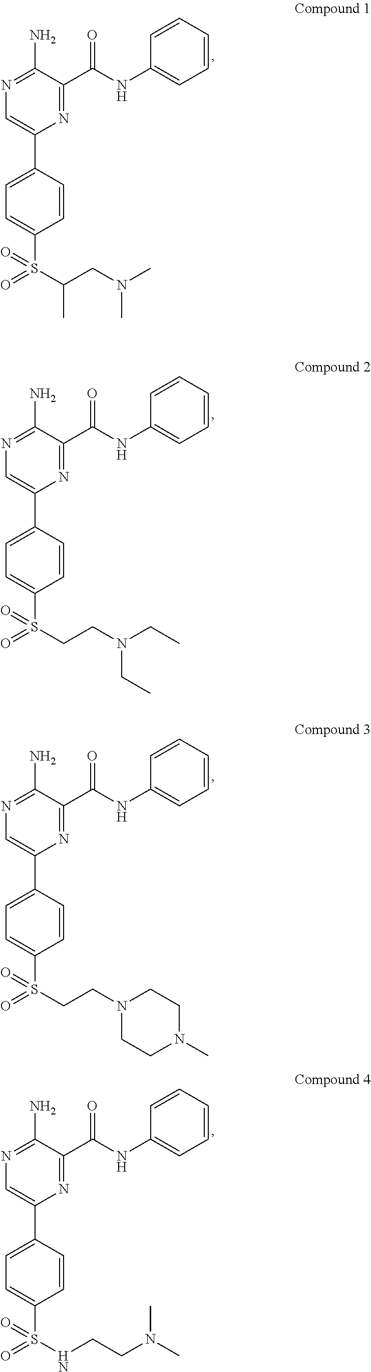 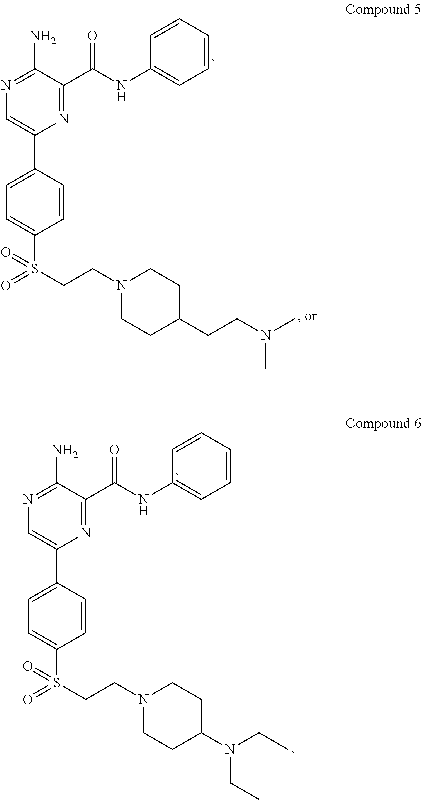 [SMILES: CN(C)CCC1CCN(CCS(=O)(=O)c2ccc(-c3cnc(N)c(C(=O)Nc4ccccc4)n3)cc2)CC1 ; CCN(CC)C1CCN(CCS(=O)(=O)c2ccc(-c3cnc(N)c(C(=O)Nc4ccccc4)n3)cc2)CC1]
or a pharmaceutically acceptable salt thereof.

47. The composition of any one of claims 39 and 42-46, wherein the phospholipid is 1,2-distearoyl-sn-glycero-3-phosphocholine (DSPC).

48. The composition of any one of claims 39, 41, and 43-46, wherein the PEG-lipid conjugate is PEG (2000)-distearoylglycerol (PEG-DSG).

49. The composition of any one of claims 40, 41, 47 and 48, wherein the liposome comprises about 50 mol % to about 70 mol % of DSPC.

50. The composition of claim 49 wherein the liposome comprises about 55 mol %, about 56 mol %, about 57 mol %, about 58 mol %, about 59 mol %, about 60 mol %, about 61 mol %, about 62 mol %, about 63 mol %, about 64 mol %, or about 65 mol % of DSPC.

51. The composition of any one of claims 39-50, wherein the liposome comprises about 30 mol % to about 50 mol % of cholesterol.

52. The composition of any one of claims 39-51, wherein the liposome comprises about 35 mol %, about 36 mol %, about 37 mol %, about 38 mol %, about 39 mol %, about 40 mol %, about 41 mol %, about 42 mol %, about 43 mol %, about 44 mol %, or about 45 mol % of cholesterol.

53. The composition of any one of claims 48-52, wherein the liposome comprises about 0.1 mol % to about 5 mol % of PEG-DSG.

54. The composition of claim 53, wherein the liposome comprises about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.6 mol %, about 0.7 mol %, about 0.8 mol %, about 0.9 mol %, about 1.0 mol %, about 1.5 mol %, about 2.0 mol %, about 2.5 mol %, about 3.0 mol %, about 3.5 mol %, about 4.0 mol %, about 4.5 mol %, or about 5.0 mol % of PEG-DSG.

55. The composition of any one of claims 48-52 wherein the liposome comprises about 0.1 mol % to about 2.5 mol % PEG-DSG.

56. The composition of claim 48-54, wherein the liposome comprises about 0.1 mol % to about 2 mol % PEG-DSG.

57. The composition of any one of claims 42-56, wherein the liposome comprises about 0.1 mol % to about 0.5 mol % PEG-DSG.

58. The composition of any one of claims 42-56, wherein the liposome comprises DSPC, cholesterol, and PEG-DSG in molar ratio of about 58:40:2 or about 59.75:40:0.25.

59. The composition of any one of claims 39-58, wherein the composition is stable for at least 6 months.

60. A pharmaceutical combination comprising a composition of any one of claims 39-59 and another pharmaceutically active agent.

61. The pharmaceutical combination of claim 60, wherein the other pharmaceutically active agent is an anti-cancer agent.

62. The composition of claim 60 or 61, wherein the other pharmaceutically active agent is irinotecan or topotecan.

63. The composition of claim 60 or 61, wherein the other pharmaceutically active agent is a platinum agent.

64. The composition of claim 63, wherein the platinum agent is cisplatin or carboplatin.

65. The composition of claim 60 or 61, wherein the other pharmaceutically active agent is gemcitabine.
